(19) United States
(12) Patent Application Publication
(10) Pub. No.: US 2021/0332036 A1
(43) Pub. Date: Oct. 28, 2021

(54) PYRIMIDINE AND PYRAZINE HDAC1,2 INHIBITORS

(71) Applicant: REGENACY PHARMACEUTICALS, LLC, Waltham, MA (US)

(72) Inventors: John H. VAN DUZER, Concord, MA (US); Ralph MAZITSCHEK, Weston, MA (US); Charles BLUM, Lexington, MA (US); Matthew B. JARPE, Quincy, MA (US)

(21) Appl. No.: 17/281,078

(22) PCT Filed: Oct. 9, 2019
(86) PCT No.: PCT/US2019/055397

§ 371 Date: Mar. 29, 2021

Related U.S. Application Data
(60) Provisional application No. 62/743,885, filed on Oct. 10, 2018.

(51) Int. Cl. C07D 409/12; C07D 295/145; C07D 417/12; C07D 405/12; C07D 409/14; C07D 401/04; C07D 403/04; C07D 417/14

(52) U.S. Cl. CPC C07D 409/12; C07D 295/145; C07D 417/12; C07D 417/14; C07D 409/14; C07D 401/04; C07D 403/04; C07D 405/12

(57) ABSTRACT

Provided herein are compounds, pharmaceutical compositions comprising such compounds, and methods of using such compounds to treat diseases or disorders associated with HDAC1 and/or HDAC2 activity.

### RELATED APPLICATION

This application claims priority to U.S. Provisional Application No. 62/743,885 filed on Oct. 10, 2018, the contents of which are incorporated in their entirety.

### BACKGROUND

A biological target of interest is histone deacetylase (HDAC) (see, for example, a discussion of the use of inhibitors of histone deacetylases for the treatment of cancer: Marks et al. Nature Reviews Cancer 2001, 7, 194; Johnstone et al. Nature Reviews Drug Discovery 2002, 287). Post-translational modification of proteins through acetylation and deacetylation of lysine residues plays a critical role in regulating their cellular functions. HDACs are zinc hydrolases that modulate gene expression through deacetylation of the N-acetyl-lysine residues of histone proteins and other transcriptional regulators (Hassig et al. Curr. Opin. Chem. Biol. 1997, 1, 300-308). HDACs participate in neuronal pathways that control synapse formation, and HDACs play a role in learning and memory (Guan et al, Nature, 2009, 459(7243) 55-60;

Eleven human HDACs, which use Zn as a cofactor, have been identified (Taunton et al. Science 1996, 272, 408-411; Yang et al. J. Biol. Chem. 1997, 272, 28001-28007. Grozinger et al. Proc. Natl. Acad. Sci. U.S.A. 1999, 96, 4868-4873; Kao et al. Genes Dev. 2000, 14, 55-66. Hu et al. J. Biol. Chem. 2000, 275, 15254-15264; Zhou et al. Proc. Natl. Acad. Sci U.S.A. 2001, 98, 10572-10577; Venter et al. Science 2001, 291, 1304-1351) and these members fall into three classes (class I, II, and IV) based on sequence homology to their yeast orthologues (O. Witt et al. Cancer Letters, 2009, 277, 8-21). Class I HDACs include HDAC1, HDAC2, HDAC3, and HDAC8, and are referred to as “classical” HDACs, which implies a catalytic pocket with a Zn²⁺ ion at its base.

There remains a need for preparing structurally diverse HDAC inhibitors, particularly ones that are potent and/or selective inhibitors of particular classes of HDACs and individual HDACs.

### SUMMARY

Provided herein are compounds and methods of using these compounds to treat disorders related to HDAC1 or HDAC2 function, including cognitive dysfunction and neurodegeneration.

In an aspect, provided herein are compounds of Formula I:

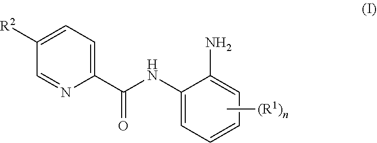

or a pharmaceutical acceptable salt thereof;

wherein:

R¹ is selected from the group consisting of aryl, halo, and heteroaryl, wherein aryl is optionally substituted with R³;

R² is selected from the group consisting of heterocycloalkyl, OR⁴, and N(H)R⁴, wherein heterocycloalkyl is optionally substituted with R⁴;

R³ is independently, at each occurrence, C₁-C₄ alkyl or —OSO₂N(H)C₁-C₄ alkyl;

R⁴ is independently, at each occurrence, selected from the group consisting of C₁-C₄ alkyl, —(C₁-C₄ alkyl)-O—(C₁-C₄ alkyl), —C(O)—C₁-C₄ alkyl, and -heterocycloalkyl-C(O)-heterocycloalkyl, wherein C₁-C₄ alkyl is optionally substituted with —OH; and

n is 1 or 2.

In another aspect, provided herein are compounds of Formula II:

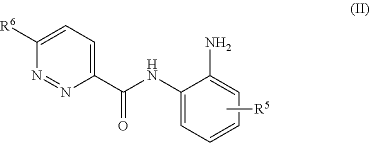

or a pharmaceutical acceptable salt thereof;

wherein:

R⁵ is aryl or heteroaryl; and

R⁶ is heterocycloalkyl or —O—(C₁-C₄ alkyl)-O—(C₁-C₄ alkyl).

In an embodiment of Formula II, R⁵ is selected from the group consisting of phenyl and thienyl; and R⁶ is selected from the group consisting of piperazine and O—(C₁-C₂ alkyl)-O-methyl.

In yet another aspect, provided herein are compounds of Formula III:

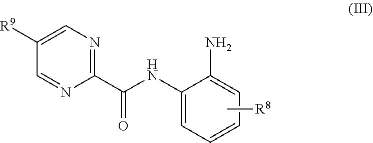

or a pharmaceutically acceptable salt thereof;

wherein:

R⁸ is aryl or heteroaryl;

R⁹ is N(H)R¹⁰ or heterocycloalkyl, wherein heterocycloalkyl is optionally substituted with C₁-C₄ alkyl;

R¹⁰ is —(C₁-C₄ alkyl)-O—(C₁-C₄ alkyl), wherein C₁-C₄ alkyl is optionally substituted with heteroaryl.

In still another aspect, provided herein is a compound of Formula IV:

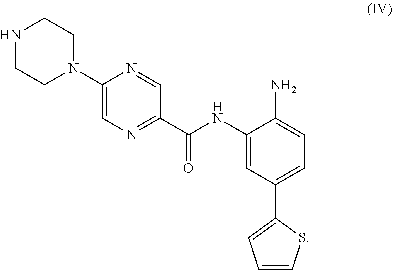 [SMILES: Nc1ccc(-c2cccs2)cc1NC(=O)c1cnc(N2CCNCC2)cn1]

In another aspect, provided herein are pharmaceutical compositions comprising any of the compounds described herein, or a pharmaceutically acceptable salt thereof, together with a pharmaceutically acceptable carrier.

In an aspect, provided herein are methods of inhibiting the activity of HDAC1 and/or HDAC2 in a subject of need thereof, comprising administering to the subject any of the compounds or compositions described herein.

In another aspect, provided herein are methods of treating a disease mediated by HDAC1 and/or HDAC2 in a subject of need thereof, comprising administering to the subject a therapeutically effective amount of any of the compounds or compositions described herein.

### DETAILED DESCRIPTION

Provided herein are compounds, e.g., the compounds of Formulae I, II, Ill, and IV, or pharmaceutically acceptable salts thereof, that are useful in the treatment of cancer, neurological disorders, myelodysplastic syndrome, or hemoglobinopathy in a subject in need thereof.

In a non-limiting aspect, these compounds can inhibit histone deacetylases. In a particular embodiment, the compounds provided herein are considered HDAC1 and/or HDAC2 inhibitors. As such, in one aspect, the compounds provided herein are useful in the treatment of cancer, myelodysplastic syndrome or hemoglobinopathy in a subject by acting as a HDAC1 and/or HDAC2 inhibitor.

### Definitions

Listed below are definitions of various terms used to describe this invention. These definitions apply to the terms as they are used throughout this specification and claims, unless otherwise limited in specific instances, either individually or as part of a larger group.

Unless defined otherwise, all technical and scientific terms used herein generally have the same meaning as commonly understood by one of ordinary skill in the art to which this invention belongs. Generally, the nomenclature used herein and the laboratory procedures in cell culture, molecular genetics, organic chemistry, and peptide chemistry are those well-known and commonly employed in the art.

As used herein, the articles “a” and “an” refer to one or to more than one (i.e., to at least one) of the grammatical object of the article. By way of example, “an element” means one element or more than one element. Furthermore, use of the term “including” as well as other forms, such as “include,” “includes,” and “included,” is not limiting.

As used herein, the term “about” will be understood by persons of ordinary skill in the art and will vary to some extent on the context in which it is used. As used herein when referring to a measurable value such as an amount, a temporal duration, and the like, the term “about” is meant to encompass variations of ±20% or ±10%, including ±5%, ±1%, and ±0.1% from the specified value, as such variations are appropriate to perform the disclosed methods.

The term “treat,” “treated,” “treating,” or “treatment” includes the diminishment or alleviation of at least one symptom associated or caused by the state, disorder or disease being treated. In certain embodiments, the treatment comprises bringing into contact with HDAC1 and/or HDAC2 an effective amount of a compound of the invention for conditions related to cancers, hemoglobinopathies, or myelodysplastic syndrome.

As used herein, the term “prevent” or “prevention” means no disorder or disease development if none had occurred, or no further disorder or disease development if there had already been development of the disorder or disease. Also considered is the ability of one to prevent some or all of the symptoms associated with the disorder or disease.

As used herein, the term “patient,” “individual,” or “subject” refers to a human or a non-human mammal. Non-human mammals include, for example, livestock and pets, such as ovine, bovine, porcine, canine, feline and marine mammals. Preferably, the patient, subject, or individual is human.

As used herein, the terms “effective amount,” “pharmaceutically effective amount,” and “therapeutically effective amount” refer to a nontoxic but sufficient amount of an agent to provide the desired biological result. That result may be reduction or alleviation of the signs, symptoms, or causes of a disease, or any other desired alteration of a biological system. An appropriate therapeutic amount in any individual case may be determined by one of ordinary skill in the art using routine experimentation.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the term “pharmaceutically acceptable salt” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present invention include the conventional non-toxic salts of the parent compound formed, for example, from non-toxic inorganic or organic acids. The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. The phrase “pharmaceutically acceptable salt” is not limited to a mono, or 1:1, salt. For example, “pharmaceutically acceptable salt” also includes bis-salts, such as a bis-hydrochloride salt. Lists of suitable salts are found in Remington's Pharmaceutical Sciences, 17th ed., Mack Publishing Company, Easton, Pa., 1985, p. 1418 and Journal of Pharmaceutical Science, 66, 2 (1977), each of which is incorporated herein by reference in its entirety.

As used herein, the term “composition” or “pharmaceutical composition” refers to a mixture of at least one compound useful within the invention with a pharmaceutically acceptable carrier. The pharmaceutical composition facilitates administration of the compound to a patient or subject. Multiple techniques of administering a compound exist in the art including, but not limited to, intravenous, oral, aerosol, parenteral, ophthalmic, pulmonary, and topical administration.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations.

As used herein, “pharmaceutically acceptable carrier” also includes any and all coatings, antibacterial and antifungal agents, and absorption delaying agents, and the like that are compatible with the activity of the compound useful within the invention, and are physiologically acceptable to the patient. Supplementary active compounds may also be incorporated into the compositions. The “pharmaceutically acceptable carrier” may further include a pharmaceutically acceptable salt of the compound useful within the invention. Other additional ingredients that may be included in the pharmaceutical compositions used in the practice of the invention are known in the art and described, for example, in Remington's Pharmaceutical Sciences (Genaro, Ed., Mack Publishing Co., 1985, Easton, Pa.), which is incorporated herein by reference.

The term “HDAC” refers to histone deacetylases, which are enzymes that remove the acetyl groups from the lysine residues in core histones, thus leading to the formation of a condensed and transcriptionally silenced chromatin. There are currently 18 known histone deacetylases, which are classified into four groups. Class I HDACs, which include HDAC1, HDAC2, HDAC3, and HDAC8, are related to the yeast RPD3 gene. Class II HDACs, which include HDAC4, HDAC5, HDAC6, HDAC7, HDAC9, and HDAC10, are related to the yeast Hda1 gene. Class III HDACs, which are also known as the sirtuins are related to the Sir2 gene and include SIRT1-7. Class IV HDACs, which contains only HDAC11, has features of both Class I and II HDACs. The term “HDAC” refers to any one or more of the 18 known histone deacetylases, unless otherwise specified.

As used herein, the term “alkyl,” by itself or as part of another substituent means, unless otherwise stated, a straight or branched chain hydrocarbon having the number of carbon atoms designated (i.e., C₁-C₆-alkyl means an alkyl having one to six carbon atoms) and includes straight and branched chains. In an embodiment, C₁-C₆ alkyl groups are provided herein. Examples include methyl, ethyl, propyl, isopropyl, butyl, isobutyl, tert-butyl, pentyl, neopentyl, and hexyl. Other examples of C₁-C₆-alkyl include ethyl, methyl, isopropyl, isobutyl, n-pentyl, and n-hexyl.

As used herein, the term “halo” or “halogen” alone or as part of another substituent means, unless otherwise stated, a fluorine, chlorine, bromine, or iodine atom, preferably, fluorine, chlorine, or bromine, more preferably, fluorine or chlorine.

As used herein, the term “heterocycloalkyl” means a non-aromatic carbocyclic system containing 1, 2, 3 or 4 heteroatoms selected independently from N, O, and S and having 1, 2 or 3 rings wherein such rings may be fused, wherein fused is defined above. Heterocycloalkyl also includes bicyclic structures that may be bridged or spirocyclic in nature with each individual ring within the bicycle varying from 3-8 atoms, and containing 0, 1, or 2 N, O, or S atoms. The term “heterocycloalkyl” includes cyclic esters (i.e., lactones) and cyclic amides (i.e., lactams) and also specifically includes, but is not limited to, epoxidyl, oxetanyl, tetrahydrofuranyl, tetrahydropyranyl (i.e., oxanyl), pyranyl, dioxanyl, aziridinyl, azetidinyl, pyrrolidinyl, 2,5-dihydro-1H-pyrrolyl, oxazolidinyl, thiazolidinyl, piperidinyl, morpholinyl, piperazinyl, thiomorpholinyl, 1,3-oxazinanyl, 1,3-thiazinanyl, 2-azabicyclo[2.1.1]-hexanyl, 5-azabicyclo[2.1.1]hexanyl, 6-azabicyclo[3.1.1] heptanyl, 2-azabicyclo-[2.2.1]heptanyl, 3-azabicyclo[3.1.1]heptanyl, 2-azabicyclo[3.1.1]heptanyl, 3-aza-bicyclo[3.1.0]hexanyl, 2-azabicyclo[3.1.0]hexanyl, 3-azabicyclo[3.2.1]octanyl, 8-azabicyclo[3.2.1]octanyl, 3-oxa-7-azabicyclo[3.3.1]nonanyl, 3-oxa-9-azabicyclo-[3.3.1]nonanyl, 2-oxa-5-azabicyclo[2.2.1]heptanyl, 6-oxa-3-azabicyclo[3.1.1]heptanyl, 2-azaspiro[3.3]heptanyl, 2-oxa-6-azaspiro[3.3]heptanyl, 2-oxaspiro[3.3]heptanyl, 2-oxaspiro[3.5]nonanyl, 3-oxaspiro[5.3]nonanyl, and 8-oxabicyclo[3.2.1]octanyl. In an embodiment, C₂-C₇ heterocycloalkyl groups are provided herein.

As used herein, the term “aromatic” refers to a carbocycle or heterocycle with one or more polyunsaturated rings and having aromatic character, i.e., having (4n+2) delocalized π (pi) electrons, where n is an integer.

As used herein, the term “aryl” means an aromatic carbocyclic system containing 1, 2 or 3 rings, wherein such rings may be fused, wherein fused is defined above. If the rings are fused, one of the rings must be fully unsaturated and the fused ring(s) may be fully saturated, partially unsaturated or fully unsaturated. The term “aryl” includes, but is not limited to, phenyl, naphthyl, indanyl, and 1,2,3,4-tetrahydro-naphthalenyl. In some embodiments, aryl groups have 6 carbon atoms. In some embodiments, aryl groups have from six to ten carbon atoms. In some embodiments, aryl groups have from six to sixteen carbon atoms. In an embodiment, 6-10 membered aryl groups are provided herein.

As used herein, the term “heteroaryl” means an aromatic carbocyclic system containing 1, 2, 3, or 4 heteroatoms selected independently from N, O, and S and having 1, 2, or 3 rings wherein such rings may be fused, wherein fused is defined above. The term “heteroaryl” includes, but is not limited to, furanyl, thiophenyl, oxazolyl, thiazolyl, imidazolyl, pyrazolyl, triazolyl, tetrazolyl, isoxazolyl, isothiazolyl, oxadiazolyl, thiadiazolyl, pyridinyl, pyridazinyl, pyrimidinyl, pyrazinyl, imidazo[1,2-a]pyridinyl, pyrazolo[1,5-a]pyridinyl, 5,6,7,8-tetrahydroisoquinolinyl, 5,6,7,8-tetrahydroquinolinyl, 6,7-dihydro-5H-cyclopenta[b]pyridinyl, 6,7-dihydro-5H-cyclopenta[c]pyridinyl, 1,4,5,6-tetrahydrocyclopenta[c]pyrazolyl, 2,4,5,6-tetrahydrocyclopenta[c]pyrazolyl, 5,6-dihydro-4H-pyrrolo[1,2-b]pyrazolyl, 6,7-dihydro-5H-pyrrolo[1,2-b][1,2,4]triazolyl, 5,6,7,8-tetrahydro-[1,2,4]triazolo[1,5-a]pyridinyl, 4,5,6,7-tetrahydropyrazolo[1,5-a]pyridinyl, 4,5,6,7-tetrahydro-1H-indazolyl and 4,5,6,7-tetrahydro-2H-indazolyl. In an embodiment, 5-10 membered heteroaryl groups are provided herein.

It is to be understood that if an aryl, heteroaryl, cycloalkyl, or heterocycloalkyl moiety may be bonded or otherwise attached to a designated moiety through differing ring atoms (i.e., shown or described without denotation of a specific point of attachment), then all possible points are intended, whether through a carbon atom or, for example, a trivalent nitrogen atom. For example, the term “pyridinyl” means 2-, 3- or 4-pyridinyl, the term “thienyl” means 2- or 3-thioenyl, and so forth.

As used herein, the term “substituted” means that an atom or group of atoms has replaced hydrogen as the substituent attached to another group.

As used herein, the term “optionally substituted” means that the referenced group may be substituted or unsubstituted. In one embodiment, the referenced group is optionally substituted with zero substituents, i.e., the referenced group is unsubstituted.

### Compounds

In an aspect, provided herein are compounds of Formula I:

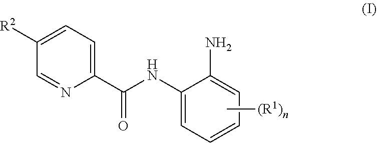

or a pharmaceutical acceptable salt thereof;

wherein:

R¹ is selected from the group consisting of aryl, halo, and heteroaryl, wherein aryl is optionally substituted with R³;

R² is selected from the group consisting of heterocycloalkyl, OR⁴, and N(H)R⁴, wherein heterocycloalkyl is optionally substituted with R⁴;

R³ is C₁-C₄ alkyl or —OSO₂N(H)C₁-C₄ alkyl;

R⁴ is independently, at each occurrence, selected from the group consisting of C₁-C₄ alkyl, —(C₁-C₄ alkyl)-O—(C₁-C₄ alkyl), —C(O)—C₁-C₄ alkyl, and -heterocycloalkyl-C(O)-heterocycloalkyl, wherein C₁-C₄ alkyl is optionally substituted with —OH; and

n is 1 or 2.

In an embodiment, R¹ is selected from the group consisting of halo, phenyl, thiazole, and thienyl, wherein phenyl is optionally substituted with R³; R² is selected from the group consisting of heterocycloalkyl, OR⁴, and N(H)R⁴, wherein heterocycloalkyl is optionally substituted with R⁴; R³ is C₁-C₄ alkyl or —OSO₂N(H)C₁-C₄ alkyl; R⁴ is independently, at each occurrence, selected from the group consisting of C₁-C₄ alkyl, —(C₁-C₄ alkyl)-O—(C₁-C₄ alkyl), —C(O)—C₁-C₄ alkyl, and -heterocycloalkyl-C(O)-hetero-cycloalkyl, wherein C₁-C₄ alkyl is optionally substituted with —OH; and n is 1 or 2.

In another embodiment, R¹ is selected from the group consisting of halo, phenyl, thienyl, and thiazole, wherein phenyl is optionally substituted with R³; R² is selected from the group consisting of piperazine, OR⁴, and N(H)R⁴, wherein piperazine is optionally substituted with R⁴; R³ methyl or —OSO₂N(H)Me; R⁴ is independently, at each occurrence, selected from the group consisting of C₁-C₃ alkyl, —(C₁-C₂ alkyl)-O-methyl, —C(O)-methyl, and -piperadine-C(O)-piperazine, wherein C₁-C₃ alkyl is optionally substituted with —OH; and n is 1 or 2.

In yet another embodiment, R¹ is halo. In still another embodiment, R¹ is phenyl. In an embodiment, R¹ is thienyl. In another embodiment, R¹ is thiazole.

In an embodiment, R² is piperazine, wherein piperazine is optionally substituted with R⁴. In another embodiment, R² is OR⁴. In yet another embodiment, R² is N(H)R⁴.

In yet another embodiment, R³ is methyl. In still another embodiment, R³ is —H. In yet another embodiment, R³ is methyl. In still another embodiment, R³ is —OSO₂N(H)Me.

In still another embodiment, R⁴ is C₁-C₃ alkyl, wherein C₁-C₃ alkyl is optionally substituted with —OH. In an embodiment, R⁴ is —(C₁-C₂ alkyl)-O-methyl. In another embodiment, R⁴ is —C(O)-methyl. In yet another embodiment, R⁴ is -piperadine-C(O)-piperazine.

In an embodiment, n is 1. In another embodiment, n is 2.

In another embodiment, aryl is a 6-10 membered aryl. In yet another embodiment, heteroaryl is a 5-10 membered heteroaryl. In still another embodiment, heterocycloalkyl is a 3-10 membered heterocycloalkyl.

In another aspect, provided herein are compounds of Formula II:

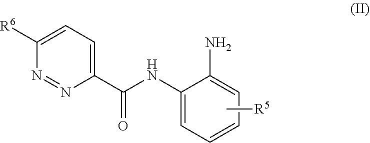

or a pharmaceutical acceptable salt thereof;

wherein:

R⁵ is aryl or heteroaryl; and

R⁶ is heterocycloalkyl or O—(C₁-C₄ alkyl)-O—(C₁-C₄ alkyl).

In an embodiment of Formula II, R⁵ is phenyl or thienyl; and R⁶ is piperazine or O—(C₁-C₂ alkyl)-O-methyl.

In another embodiment R⁵ is phenyl. In yet another embodiment R⁵ is thienyl.

In still another embodiment, R⁶ is piperazine. In an embodiment, R⁶ is O—(C₁-C₂ alkyl)-O-methyl. In another embodiment, R⁶ is OCH₂CH₂OCH₃. In yet another embodiment, R⁶ is OCH₂OCH₃.

In another embodiment, aryl is a 6-10 membered aryl. In yet another embodiment, heteroaryl is a 5-10 membered heteroaryl. In still another embodiment, heterocycloalkyl is a 3-10 membered heterocycloalkyl.

In yet another aspect, provided herein are compounds of Formula III:

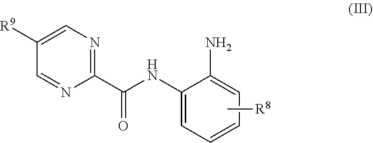

or a pharmaceutically acceptable salt thereof;

wherein:

R⁸ is aryl or heteroaryl;

R⁹ is N(H)R¹⁰ or heterocycloalkyl, wherein heterocycloalkyl is substituted with C₁-C₄ alkyl;

R¹⁰ is —(C₁-C₄alkyl)-O—(C₁-C₄ alkyl), wherein C₁-C₄ alkyl is optionally substituted with heteroaryl.

In an embodiment, R⁸ is selected from the group consisting of furanyl, phenyl, and theinyl; R⁹ is N(H)R¹⁰ or heterocycloalkyl, wherein heterocycloalkyl is optionally substituted with C₁-C₄ alkyl; and R¹⁰ is —(C₁-C₄ alkyl)-O—(C₁-C₄ alkyl), wherein C₁-C₄ alkyl is optionally substituted with heteroaryl.

In another embodiment, R⁸ is selected from the group consisting of furanyl, phenyl, and thienyl; R⁹ is N(H)R¹⁰ or piperazine, wherein piperazine is optionally substituted with methyl; and R¹⁰ is —(C₁-C₂ alkyl)-O—(C₁-C₂ alkyl), wherein C₁-C₂alkyl is optionally substituted with pyridine.

In yet another embodiment, R⁸ is furanyl. In still another embodiment, R⁸ is phenyl. In an embodiment, R⁸ is thienyl.

In another embodiment, R⁹ is N(H)R¹⁰. In yet another embodiment, R⁹ is piperazine, wherein piperazine is optionally substituted with methyl.

In still another embodiment, R¹⁰ is —(C₁-C₂ alkyl)-O—(C₁-C₂ alkyl), wherein C₁-C₂ alkyl is optionally substituted with pyridine.

In another embodiment, aryl is a 6-10 membered aryl. In yet another embodiment, heteroaryl is a 5-10 membered heteroaryl. In still another embodiment, heterocycloalkyl is a 3-10 membered heterocycloalkyl.

In still another aspect, provided herein is a compound of Formula IV:

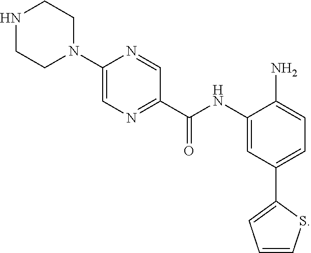 [SMILES: Nc1ccc(-c2cccs2)cc1NC(=O)c1cnc(N2CCNCC2)cn1]

In still another embodiment, the compound of Formula I, Formula II, Formula III, or Formula IV is selected from the group consisting of the compounds in Table 1.

TABLE 1

| Structure | Compound No. |
|---|---|
| 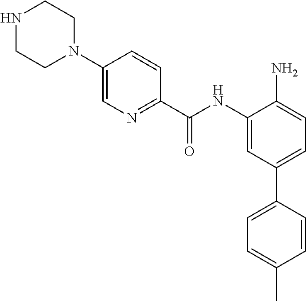 [SMILES: Cc1ccc(-c2ccc(N)c(NC(=O)c3ccc(N4CCNCC4)cn3)c2)cc1] | 001 |
| 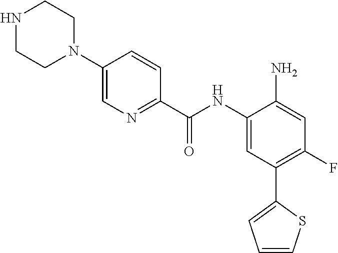 [SMILES: Nc1cc(F)c(-c2cccs2)cc1NC(=O)c1ccc(N2CCNCC2)cn1] | 002 |
| 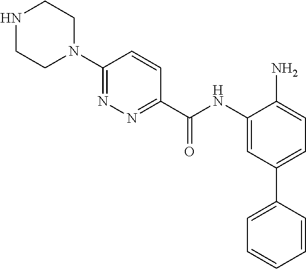 [SMILES: Nc1ccc(-c2ccccc2)cc1NC(=O)c1ccc(N2CCNCC2)nn1] | 003 |
| 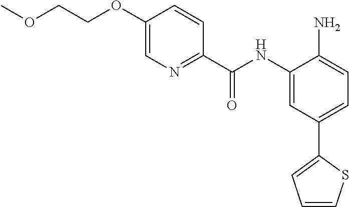 [SMILES: COCCOc1ccc(C(=O)Nc2cc(-c3cccs3)ccc2N)nc1] | 004 |
| 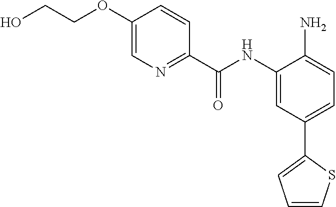 [SMILES: Nc1ccc(-c2cccs2)cc1NC(=O)c1ccc(OCCO)cn1] | 005 |
| 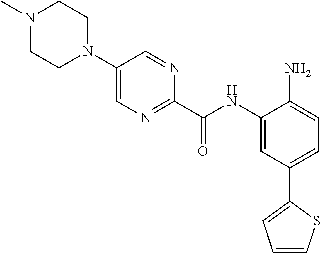 [SMILES: CN1CCN(c2cnc(C(=O)Nc3cc(-c4cccs4)ccc3N)nc2)CC1] | 006 |
| 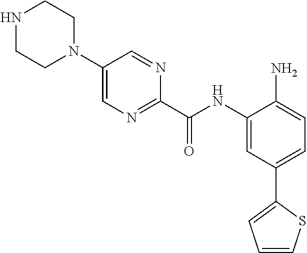 [SMILES: Nc1ccc(-c2cccs2)cc1NC(=O)c1ncc(N2CCNCC2)cn1] | 007 |
| 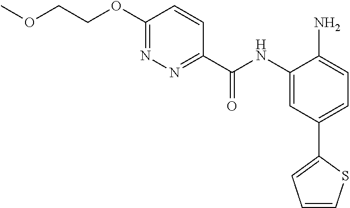 [SMILES: COCCOc1ccc(C(=O)Nc2cc(-c3cccs3)ccc2N)nn1] | 008 |
| 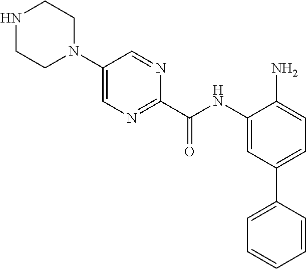 [SMILES: Nc1ccc(-c2ccccc2)cc1NC(=O)c1ncc(N2CCNCC2)cn1] | 009 |
| 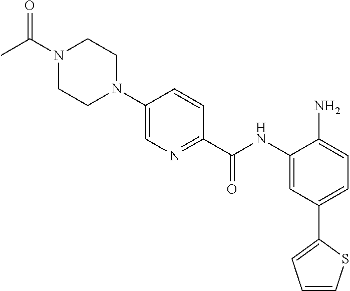 [SMILES: CC(=O)N1CCN(c2ccc(C(=O)Nc3cc(-c4cccs4)ccc3N)nc2)CC1] | 010 |
| 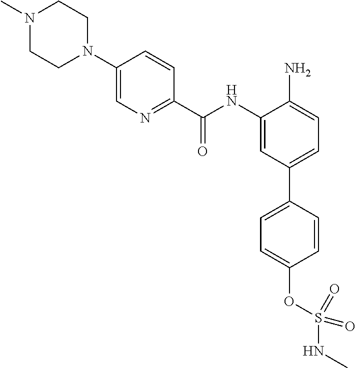 [SMILES: CNS(=O)(=O)Oc1ccc(-c2ccc(N)c(NC(=O)c3ccc(N4CCN(C)CC4)cn3)c2)cc1] | 011 |
| 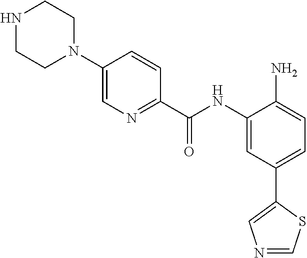 [SMILES: Nc1ccc(-c2cncs2)cc1NC(=O)c1ccc(N2CCNCC2)cn1] | 012 |
| 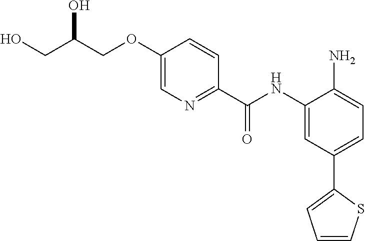 [SMILES: Nc1ccc(-c2cccs2)cc1NC(=O)c1ccc(OC[C@H](O)CO)cn1] | 013 |
| 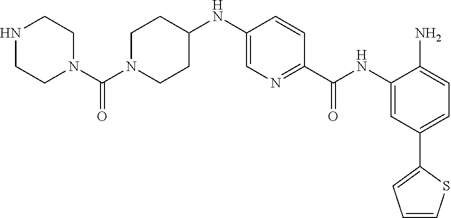 [SMILES: Nc1ccc(-c2cccs2)cc1NC(=O)c1ccc(NC2CCN(C(=O)N3CCNCC3)CC2)cn1] | 014 |
| 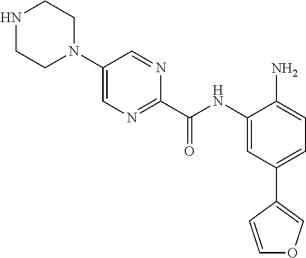 [SMILES: Nc1ccc(-c2ccoc2)cc1NC(=O)c1ncc(N2CCNCC2)cn1] | 015 |
| 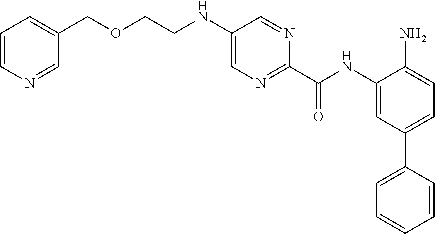 [SMILES: Nc1ccc(-c2ccccc2)cc1NC(=O)c1ncc(NCCOCc2cccnc2)cn1] | 016 |
| 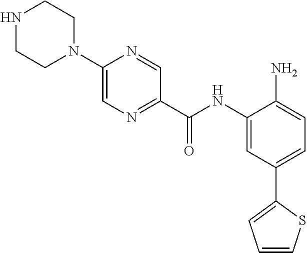 [SMILES: Nc1ccc(-c2cccs2)cc1NC(=O)c1cnc(N2CCNCC2)cn1] | 017 |

or a pharmaceutically acceptable salt thereof.

In an aspect, provided herein are pharmaceutical compositions comprising any of the compounds described herein, or a pharmaceutically acceptable salt thereof, together with a pharmaceutically acceptable carrier.

In one embodiment, the disclosed compounds may exist as tautomers. All tautomers are included within the scope of the compounds presented herein.

Compounds described herein also include isotopically-labeled compounds wherein one or more atoms is replaced by an atom having the same atomic number, but an atomic mass or mass number different from the atomic mass or mass number usually found in nature. Examples of isotopes suitable for inclusion in the compounds described herein include and are not limited to ²H, ³H, ¹¹C, ¹³C, ¹⁴C, ³⁶Cl, ¹⁸F, 123I, ¹²⁵I, ¹³N, ¹⁵N, ¹⁵O, ¹⁷O, ¹⁸O, ³²P, and ³⁵S. In another embodiment, isotopically-labeled compounds are useful in drug or substrate tissue distribution studies. In another embodiment, substitution with heavier isotopes such as deuterium affords greater metabolic stability (for example, increased in vivo half-life or reduced dosage requirements). In yet another embodiment, the compounds described herein include a ²H (i.e., deuterium) isotope.

In still another embodiment, substitution with positron emitting isotopes, such as ¹¹C, ¹⁸F, ¹⁵O and ¹³N, is useful in Positron Emission Topography (PET) studies for examining substrate receptor occupancy. Isotopically-labeled compounds are prepared by any suitable method or by processes using an appropriate isotopically-labeled reagent in place of the non-labeled reagent otherwise employed.

The specific compounds described herein, and other compounds encompassed by one or more of the Formulas described herein having different substituents are synthesized using techniques and materials described herein and as described, for example, in Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), Larock's Comprehensive Organic Transformations (VCH Publishers Inc., 1989), March, Advanced Organic Chemistry 4th Ed., (Wiley 1992); Carey and Sundberg, Advanced Organic Chemistry 4th Ed., Vols. A and B (Plenum 2000, 2001), and Green and Wuts, Protective Groups in Organic Synthesis 3rd Ed., (Wiley 1999) (all of which are incorporated by reference for such disclosure). General methods for the preparation of compounds as described herein are modified by the use of appropriate reagents and conditions, for the introduction of the various moieties found in the Formulas as provided herein.

Compounds described herein are synthesized using any suitable procedures starting from compounds that are available from commercial sources, or are prepared using procedures described herein.

### Methods of Treatment

The compounds disclosed here can be used in a method of treating a disease or condition in a subject, said method comprising administering to the subject a compound provided herein, or a pharmaceutical composition comprising the compound.

In one aspect, provided herein is a method of selectively inhibiting HDAC1 and/or HDAC2 over other HDACs (e.g., HDAC3 and HDAC6) in a subject, comprising administering to the subject a compound of Formulae I, II, Ill, or IV or any of the compounds of Table 1 or pharmaceutically acceptable salts thereof.

In an embodiment, the compound of any of the formulae herein (e.g., Formula I, II, Ill, or IV) has a selectivity for HDAC1 and/or HDAC2 of 5 to 1000 fold over other HDACs.

In another embodiment, the compound of any of the formulae herein (e.g., Formula I, II, Ill, IV) has a selectivity for HDAC1 and/or HDAC2 when tested in a HDAC enzyme assay of about 5 to 1000 fold over other HDACs.

In certain embodiments, the compound has a selectivity for HDAC1 and/or HDAC2 of 15 to 40 fold over other HDACs.

In another aspect, provided herein is a method of treating a disease mediated by HDAC1 and/or HDAC2 in a subject comprising administering to the subject a compound of Formula II, Ill, IV, or any of the compounds of Table 1.

In an aspect, the compounds are able to treat a subject suffering from or susceptible to a hemoglobinopathy. In an embodiment, the compounds are able to treat sickle-cell disease or beta-thalessemia.

In another aspect, the compounds provided herein are useful in the treatment of myelodysplastic syndromes.

In another embodiment, the disease is cancer.

In yet another embodiment, the cancer is lung cancer, colon and rectal cancer, breast cancer, prostate cancer, liver cancer, pancreatic cancer, brain cancer, kidney cancer, ovarian cancer, stomach cancer, skin cancer, bone cancer, gastric cancer, glioma, glioblastoma, neuroblastoma, hepatocellular carcinoma, papillary renal carcinoma, head and neck squamous cell carcinoma, leukemia, lymphomas, myelomas, retinoblastoma, cervical cancer, melanoma and/or skin cancer, bladder cancer, uterine cancer, testicular cancer, esophageal cancer, and solid tumors. In some embodiments, the cancer is lung cancer, colon cancer, breast cancer, neuroblastoma, leukemia, or lymphomas. In other embodiments, the cancer is lung cancer, colon cancer, breast cancer, neuroblastoma, leukemia, or lymphoma. In a further embodiment, the cancer is non-small cell lung cancer (NSCLC) or small cell lung cancer.

In still another embodiment, the cancer is glioblastoma.

In an embodiment, the cancer is a hematologic cancer, such as leukemia or lymphoma. In a certain embodiment, lymphoma is Hodgkin's lymphoma or Non-Hodgkin's lymphoma. In certain embodiments, leukemia is myeloid, lymphocytic, myelocytic, lymphoblastic, or megakaryotic leukemia.

In another embodiment, the leukemia is acute myelogenous leukemia and megakaryocytic leukemia.

In yet another aspect, provided herein is a method of treating a neurodegenerative disease in a subject in need thereof comprising administering to the subject in need thereof a therapeutically effective amount of a compound of Formula I, II, Ill, IV, a compound of Table 1, or a pharmaceutically acceptable salt thereof.

In an embodiment, the neurodegenerative disease is selected from the group consisting of Alzheimer's disease, senile dementia, vascular dementia, Parkinson's disease, Huntington's disease, frontotemporal dementia, and progressive supranuclear palsy,

In still another aspect, provided herein is a method of treating a psychological disorder in a subject in need thereof comprising administering to the subject in need thereof a therapeutically effective amount of a compound of Formula I, II, Ill, IV, a compound of Table 1, or a pharmaceutically acceptable salt thereof.

In an embodiment, the psychological disorder is selected from the group consisting of schizophrenia, major depression, substance use disorder, alcoholism, opiate addiction, cocaine addiction, and post-traumatic stress disorder.

In another aspect, provided herein is a method for treating a disease selected from the group consisting of sickle cell disease, beta thalassemia, myelodysplastic syndrome, acute myelogenous leukemia, neuroblastoma, and megakaryocytic leukemia in a subject in need thereof comprising administering to the subject in need thereof a therapeutically effective amount of a compound of Formula I, II, Ill, IV, a compound of Table 1, or a pharmaceutically acceptable salt thereof.

Thus, in another aspect, methods for the treatment of a disease mediated by HDAC1 and/or HDAC2 are provided comprising administering a therapeutically effective amount of a compound of Formula I, as described herein, to a subject in need thereof. In certain embodiments, the subject is identified as in need of such treatment. In certain embodiments, a method for the treatment of a diseases is provided comprising administering a therapeutically effective amount of a compound disclosed herein, or a pharmaceutical composition comprising a compound disclosed herein to a subject in need thereof, in such amounts and for such time as is necessary to achieve the desired result.

In certain embodiments, the method involves the administration of a therapeutically effective amount of a compound disclosed herein or a pharmaceutically acceptable salt thereof to a subject (including, but not limited to a human or animal) in need of it (including a subject identified as in need).

### Administration/Dosage/Formulations

In another aspect, provided herein is a pharmaceutical composition comprising at least one compound provided herein, together with a pharmaceutically acceptable carrier.

Actual dosage levels of the active ingredients in the pharmaceutical compositions discussed herein may be varied so as to obtain an amount of the active ingredient that is effective to achieve the desired therapeutic response for a particular patient, composition, and mode of administration, without being toxic to the patient.

In particular, the selected dosage level will depend upon a variety of factors including the activity of the particular compound employed, the time of administration, the rate of excretion of the compound, the duration of the treatment, other drugs, compounds or materials used in combination with the compound, the age, sex, weight, condition, general health and prior medical history of the patient being treated, and like factors well, known in the medical arts.

A medical doctor, e.g., physician or veterinarian, having ordinary skill in the art may readily determine and prescribe the effective amount of the pharmaceutical composition required. For example, the physician or veterinarian could begin administration of the pharmaceutical composition to dose the disclosed compound at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of the disclosed compound calculated to produce the desired therapeutic effect in association with the required pharmaceutical vehicle. The dosage unit forms are dictated by and directly dependent on (a) the unique characteristics of the disclosed compound and the particular therapeutic effect to be achieved, and (b) the limitations inherent in the art of compounding/formulating such a disclosed compound for the treatment of pain, a depressive disorder, or drug addiction in a patient.

In one embodiment, the compounds provided herein are formulated as pharmaceutical compositions using one or more pharmaceutically acceptable excipients or carriers. In one embodiment, the pharmaceutical compositions comprise a therapeutically effective amount of a disclosed compound and a pharmaceutically acceptable carrier.

Routes of administration of any of the compositions disclosed herein include oral, nasal, rectal, intravaginal, parenteral, buccal, sublingual or topical. The compounds disclosed herein may be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intragastrical, intrathecal, subcutaneous, intramuscular, intradermal, intra-arterial, intravenous, intrabronchial, inhalation, and topical administration. In one embodiment, the preferred route of administration is oral.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions are not limited to the particular formulations and compositions that are described herein.

For oral application, particularly suitable are tablets, dragees, liquids, drops, suppositories, or capsules, caplets and gel caps. The compositions intended for oral use may be prepared according to any method known in the art and such compositions may contain one or more agents selected from the group consisting of inert, non-toxic pharmaceutically excipients that are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

For parenteral administration, the disclosed compounds may be formulated for injection or infusion, for example, intravenous, intramuscular or subcutaneous injection or infusion, or for administration in a bolus dose or continuous infusion. Suspensions, solutions or emulsions in an oily or aqueous vehicle, optionally containing other formulatory agents such as suspending, stabilizing or dispersing agents may be used.

Those skilled in the art will recognize, or be able to ascertain using no more than routine experimentation, numerous equivalents to the specific procedures, embodiments, claims, and examples described herein. Such equivalents were considered to be within the scope of this disclosure and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that wherever values and ranges are provided herein, all values and ranges encompassed by these values and ranges, are meant to be encompassed within the scope of the present disclosure. Moreover, all values that fall within these ranges, as well as the upper or lower limits of a range of values, are also contemplated by the present application.

The following examples further illustrate aspects of the present disclosure. However, they are in no way a limitation of the teachings or disclosure of the present application as set forth.

### EXAMPLES

The application is further illustrated by the following examples, which should not be construed as further limiting. The practice of the present disclosure will employ, unless otherwise indicated, conventional techniques of organic synthesis, cell biology, cell culture, and molecular biology, which are within the skill of the art.

### Abbreviations

Ac acetyl
° C. degree Celsius
dba dibenzylideneacetone
DCM dichloromethane
DIPEA N,N-Diisopropylethylamine
DMAP 4-dimethylaminopyridine
DMF dimethylformamide
DMSO dimethylsulfoxide
dppf 1,1′-bis(diphenylphosphino)ferrocene
EDCI 1-ethyl-3-[3-(dimethylamino)propyl]carbodiimidhydrochloride
EA ethyl acetate
HATU O-(7-Azabenzotriazol-1-yl)-N,N,N′,N′-tetramethyluronium Hexafluorophosphate
HOAt 1-hydroxy-7-azabenzotriazole
HOBt 1-hydroxybenzotriazole
HPLC high performance liquid chromatography
m-CPBA m-chloroperoxybenzoicacid
PE petroleum ether
Ph phenyl
Py pyridine
Ruphos 2-Dicyclohexylphosphino-2′,6′-di-i-propoxy-1,1′-biphenyl
S-Phos 2-Dicyclohexylphosphino-2′,6′-dimethoxybiphenyl
TFA trifluoroacetic acid
THF tetrahydrofuran
TLC thin layer chromatography
Tol toluene or tolyl
Xantphos 4,5-Bis(diphenylphosphino)-9,9-dimethylxanthene

### Synthesis Procedures

### Example 1

### Synthesis of Compound 001

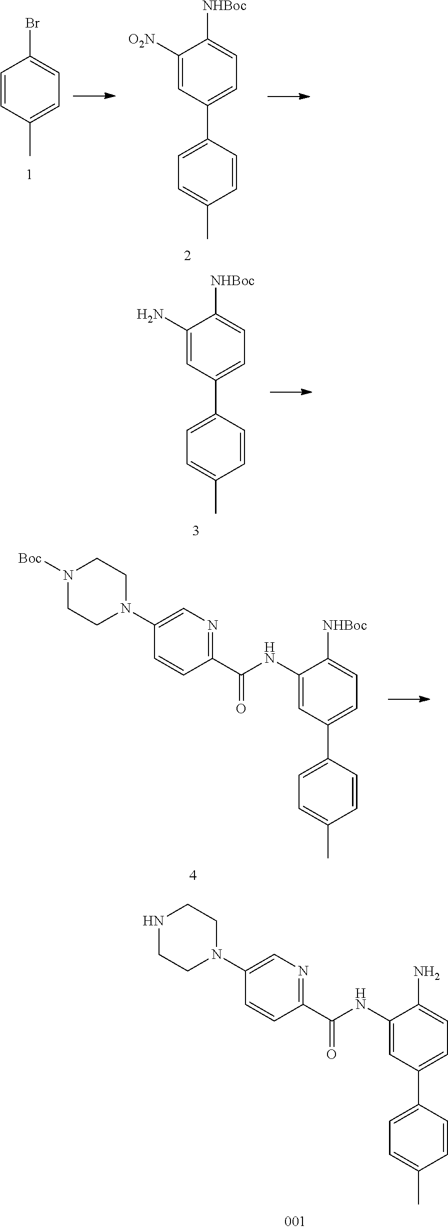

### Step 1:

Compound 1 (171 mg, 1 mmol), tert-butyl-2-nitro-4-(4,4,5,5-tetramethyl-1,3,2-dioxaborolan-2-yl) phenylcarbamate (364 mg, 1 mmol), Pd(dppf)Cl₂ (41 mg, 0.05 mmol) and K₂CO₃ (276 mg, 2 mmol) were combined in 1,4-dioxane (10 mL) and H₂O (3 mL). The mixture was stirred at 90° C. overnight. The crude material was purified by CombiFlash (PE/EA=2/1) to afford compound 2 (280 mg, 85%) as yellow solid.

### Step 2:

Compound 2 (280 mg, 0.85 mmol), FeCl₃ (15 mg, 0.09 mmol), and activated carbon (30 mg) were combined in EtOH (10 mL). The reaction mixture was heated to 60° C. and N₂H₄.H₂O (2 mL) was added dropwise. The reaction mixture was stirred for 3 h. Once the reaction was complete, the crude material was filtered, concentrated, and washed with Et₂O. Compound 3 was isolated as a white solid (211 mg, 83%).

### Step 3:

Compound 3 (100 mg, 0.34 mmol), 5-(4-(tert-butoxycarbonyl) piperazin-1-yl) picolinic acid (104 mg, 0.34 mmol), and EDCI (196 mg, 1.02 mmol) were combined in Py (5 mL). The reaction mixture was stirred at rt overnight then concentrated. The crude material was carried over to the next step (200 mg, crude).

### Step 4:

Compound 4 (200 mg, 0.34 mmol) was dissolved in DCM (4 mL). Then TFA (2 mL) was added at rt and the mixture was stirred for 2 h. Once complete, the reaction was concentrated and purified by Prep-HPLC (base method). White solid was afforded as Compound 001 (24 mg, 18%). LCMS: m/z=388.3 (M+H)⁺. ¹H NMR (400 MHz, DMSO) δ 9.88 (s, 1H), 8.37 (d, J=2.5 Hz, 1H), 7.95 (d, J=8.8 Hz, 1H), 7.88 (d, J=1.8 Hz, 1H), 7.45 (d, J=8.2 Hz, 3H), 7.27-7.17 (m, 3H), 6.89 (d, J=8.3 Hz, 1H), 4.99 (s, 2H), 3.31-3.25 (m, 4H), 2.89-2.78 (m, 4H), 2.31 (s, 3H).

### Example 2

### Synthesis of Compound 002

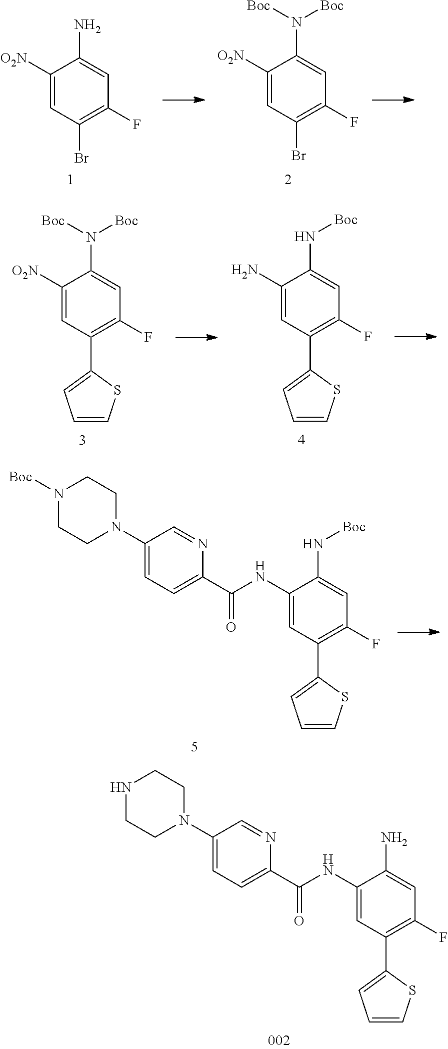 [SMILES: Nc1cc(F)c(Br)cc1[N+](=O)[O-] ; CC(C)(C)OC(=O)N(C(=O)OC(C)(C)C)c1cc(F)c(Br)cc1[N+](=O)[O-] ; CC(C)(C)OC(=O)N(C(=O)OC(C)(C)C)c1cc(F)c(-c2cccs2)cc1[N+](=O)[O-] ; CC(C)(C)OC(=O)Nc1cc(F)c(-c2cccs2)cc1N ; CC(C)(C)OC(=O)Nc1cc(F)c(-c2cccs2)cc1NC(=O)c1ccc(N2CCN(C(=O)OC(C)(C)C)CC2)cn1 ; Nc1cc(F)c(-c2cccs2)cc1NC(=O)c1ccc(N2CCNCC2)cn1]

### Step 1:

Compound 1 (699 mg, 3 mmol), Boc₂O (1.96 g, 9 mmol), and DMAP (37 mg, 0.3 mmol) were dissolved in DCM (10 mL). The reaction was stirred at rt overnight. The crude material was purified by CombiFlash (PE/EA=2/1) to afford compound 2 (1.2 g, 92%) as a white solid.

### Step 2:

Compound 2 (217 mg, 0.5 mmol), thiophen-2-ylboronic acid (64 mg, 0.5 mmol), Pd(PPh₃)₄ (30 mg, 0.025 mmol) and Cs₂CO₃ (326 mg, 1 mmol) were combined in 1,4-dioxane (10 mL) and H₂O (3 mL). The reaction mixture was stirred at 90° C. overnight and purified by CombiFlash (PE/EA=2/1) to afford compound 3 (110 mg, 50%) as white solid.

### Step 3:

Compound 3 (90 mg, 0.21 mmol), FeCl₃ (3 mg, 0.02 mmol), and activated carbon (20 mg) were combined in EtOH (10 mL). The reaction mixture was heated to 60° C. and N₂H₄.H₂O (1 mL) was added dropwise. The reaction was stirred for 3 h. Once complete, the reaction material was filtered, concentrated, and washed with Et₂O. Compound 4 was isolated as a white solid (30 mg, 48%).

### Step 4:

Compound 4 (30 mg, 0.1 mmol), 5-(4-(tert-butoxycarbonyl) piperazin-1-yl) picolinic acid (31 mg, 0.1 mmol) and EDCI (58 mg, 0.3 mmol) were dissolved in Py (3 mL). The reaction mixture was stirred at rt overnight, then concentrated and purified by CombiFlash (PE/EA=1/1) to afford compound 5 (30 mg, 52%) as white solid.

### Step 5:

Compound 5 (30 mg, 0.05 mmol) was dissolved in DCM (2 mL). Then TFA (1 mL) was added at rt and the reaction mixture was stirred for 2 h. Once the reaction was complete, the crude material was concentrated and washed with Et₂O. Yellow solid was afforded as Compound 002 (23 mg, TFA salt). LCMS: m/z=398.1 (M+H)⁺. ¹H NMR (400 MHz, DMSO) δ 9.86 (s, 1H), 9.02 (s, 2H), 8.44 (s, 1H), 8.00 (d, J=8.7 Hz, 1H), 7.74 (d, J=8.4 Hz, 1H), 7.56 (d, J=8.8 Hz, 1H), 7.48 (d, J=5.1 Hz, 1H), 7.29 (s, 1H), 7.11 (d, J=4.2 Hz, 1H), 6.70 (d, J=13.1 Hz, 1H), 3.60 (d, J=4.7 Hz, 4H), 3.28 (s, 4H).

### Example 3

### Synthesis of Compound 003

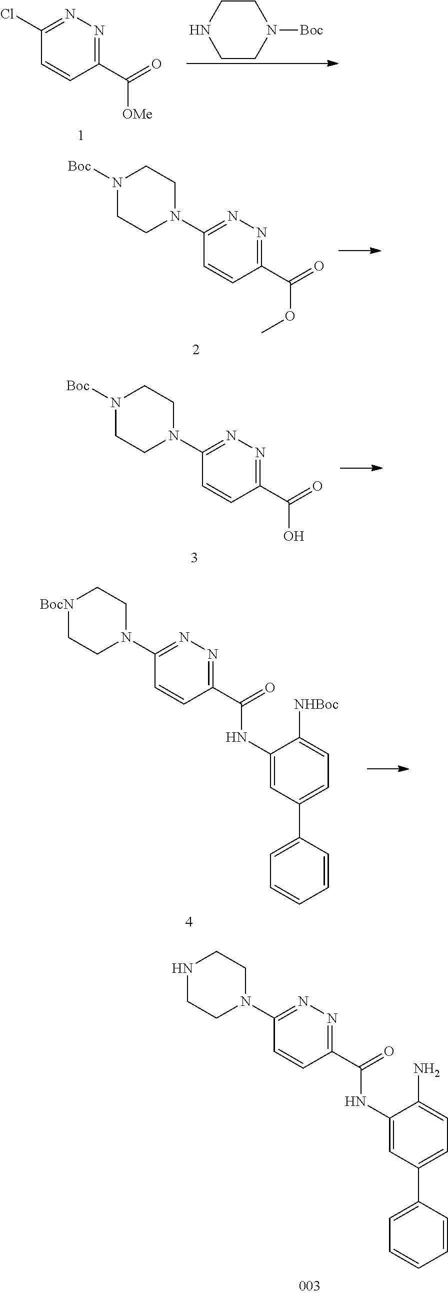 [SMILES: COC(=O)c1ccc(Cl)nn1 ; CC(C)(C)OC(=O)N1CCNCC1 ; COC(=O)c1ccc(N2CCN(C(=O)OC(C)(C)C)CC2)nn1 ; CC(C)(C)OC(=O)N1CCN(c2ccc(C(=O)O)nn2)CC1 ; CC(C)(C)OC(=O)Nc1ccc(-c2ccccc2)cc1NC(=O)c1ccc(N2CCN(C(=O)OC(C)(C)C)CC2)nn1 ; Nc1ccc(-c2ccccc2)cc1NC(=O)c1ccc(N2CCNCC2)nn1]

### Step 1:

Compound 1 (1 g, 5.81 mmol), tert-butyl piperazine-1-carboxylate (1.1 g, 5.81 mmol), and DIPEA (2.3 g, 17.43 mmol) were combined in DMF (20 mL). The reaction solution was heated to 100° C. and allowed to stir overnight. The crude material was purified via column chromatograph on silica gel (200-300 mesh, eluting with DCM/EtOAc=1:1) to afford Compound 2 as a yellow solid (1.7 g, 91%).

### Step 2:

Compound 2 (1.7 g, 5.28 mmol) and LiOH (333 mg, 7.92 mmol) were combined in MeOH (3 mL), THF (20 mL) and H₂O (5 mL). The reaction was stirred at rt overnight. The pH of the reaction was adjusted to be less than 7 using HCl (3N). Then the reaction mixture was concentrated in vacuo to afford Compound 4 as a yellow solid (2.1 g, crude).

### Step 3:

To a solution of compound 3 (100 mg, 0.33 mmol) and tert-butyl 3-aminobiphenyl-4-ylcarbamate (77 mg, 0.27 mmol) in Py (3 ml) was added EDCI (156 mg, 0.81 mmol). The reaction mixture was stirred at rt overnight then purified via column chromatograph on silica gel (200-300 mesh, eluting with petroleum ether/EtOAc=1:1) to afford Compound 4 as a white solid (85 mg, 55%).

### Step 4:

To a solution of compound 4 (85 mg, 0.15 mmol) in DCM (2 ml) at 0° C. was added TFA (1.5 mL) drop wise. The resulting mixture was stirred at rt for 1 h. After the reaction had completed, the mixture was concentrated in vacuo and washed with ether to afford Compound 003 as a gray solid (38 mg, TFA salt). LCMS: m/z=375.2 (M+H)⁺. ¹H NMR (400 MHz, DMSO) δ 10.26 (s, 1H), 9.03 (s, 2H), 8.05 (d, J=9.5 Hz, 1H), 7.80 (d, J=2.0 Hz, 1H), 7.55 (dd, J=17.0, 8.5 Hz, 3H), 7.45-7.33 (m, 3H), 7.27 (t, J=7.4 Hz, 1H), 6.97 (d, J=8.3 Hz, 1H), 4.03-3.96 (m, 4H), 3.28 (s, 4H).

### Example 4

### Synthesis of Compound 004

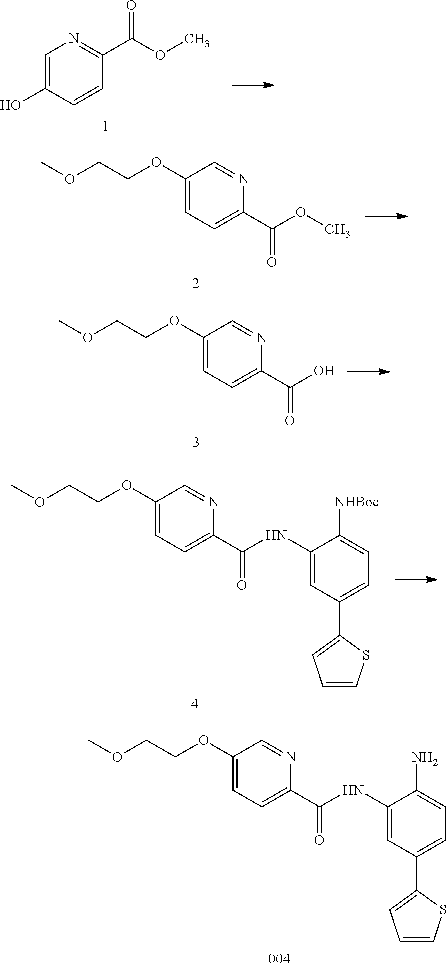

### Step 1:

Compound 1 (350 mg, 2.29 mmol), 1-chloro-2-methoxyethane (238 mg, 2.52 mmol), and K₂CO₃ (473 mg, 3.42 mmol) were combined in DMF (8 mL). The reaction was stirred at 120° C. overnight. The aqueous layer was extracted with EtOAc (3×100 mL). The combined organic layers were washed with brine (30 mL), dried over Na₂SO₄, filtered, and concentrated in vacuo to give Compound 2 (572 mg, crude).

### Step 2:

Compound 2 (572 mg, crude) and LiOH (107 mg, 2.5 mmol) were combined in THF (10 mL), CH₃OH (1.5 mL) and water (3 mL). The reaction was stirred at rt for 1 h. THF and CH₃OH were removed in vacuo. The aqueous phase was adjusted to pH 7 and concentrated in vacuo to give Compound 3 (720 mg, crude).

### Step 3:

Compound 3 (180 mg, crude), tert-butyl-(2-amino-4-(thiophen-2-yl)phenyl)carbamate (147 mg, 0.51 mmol) and EDCI (194 mg, 1.01 mmol) were combined in pyridine (4 mL). The reaction was stirred at rt overnight and concentrated in vacuo. The crude material was purified via column chromatography on silica gel (200-300 mesh, eluting with petroleum ether/EtOAc=1:1) to afford Compound 4 (175 mg, 40.8%).

### Step 4:

Compound 4 (175 mg, 0.37 mmol) and TFA (2 mL) were combined in DCM (2 mL) and stirred at rt for 1 h. The reaction mixture was concentrated in vacuo. The crude mixture was diluted with water (10 mL) and extracted with EtOAc (2×20 mL). The combined organic layers were washed with saturated NaHCO₃ (20 mL), dried over NaSO₄, filtered, and concentrated in vacuo. The crude residue was re-crystallized from water and dried by lyophilization to afford Compound 004 (54 mg, 39.2%). LCMS: m/z=370.1 (M+H)⁺. 1H NMR (400 MHz, DMSO) δ 9.96 (s, 1H), 8.42 (d, J=2.8 Hz, 1H), 8.11 (d, J=8.7 Hz, 1H), 7.82 (d, J=2.0 Hz, 1H), 7.63 (dd, J=8.7, 2.9 Hz, 1H), 7.40-7.34 (m, 1H), 7.30-7.23 (m, 2H), 7.06 (dd, J=5.0, 3.6 Hz, 1H), 6.85 (d, J=8.3 Hz, 1H), 5.12 (s, 2H), 4.34-4.27 (m, 2H), 3.75-3.68 (m, 2H), 3.33 (d, J=1.7 Hz, 3H).

### Example 5

### Synthesis of Compound 006

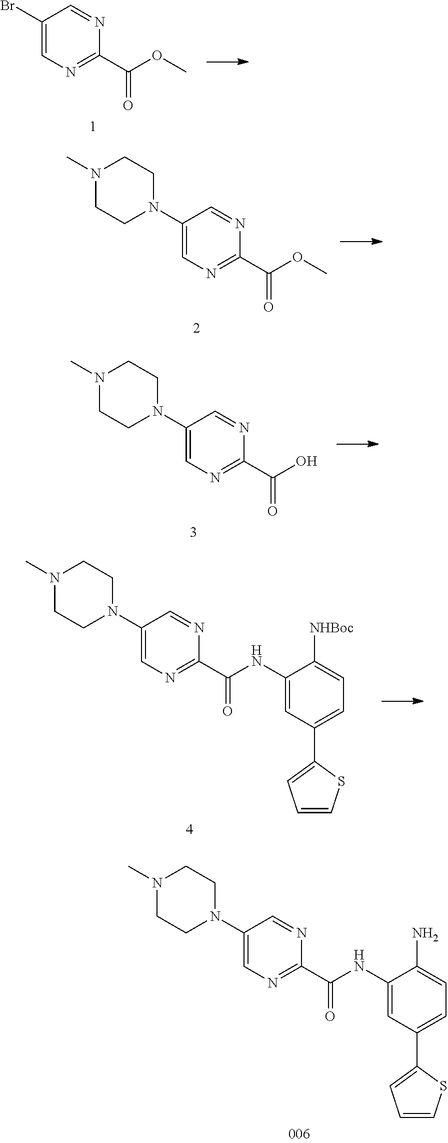

### Step 1:

Compound 1 (217 mg, 1 mmol), 1-methylpiperazine (200 mg, 2 mmol), Pd₂(dba)₃(46 mg, 0.05 mmol) and RuPhos (47 mg, 0.1 mmol) were added into toluene (10 mL). The mixture was stirred at 95° C. overnight then concentrated and carried over to the next step (300 mg, crude).

### Step 2:

Compound 2 (300 mg, 1.27 mmol) and LiOH.H₂O (100 mg, 2.54 mmol) were added into MeOH (10 mL). The mixture was heated to 60° C. and stirred for 3 h then concentrated and carried over to the next step (300 mg, crude).

### Step 3:

Compound 3 (300 mg, 1.35 mmol), tert-butyl 2-amino-4-(thiophen-2-yl) phenylcarbamate (290 mg, 1 mmol) and EDCI (576 mg, 3 mmol) were added into Py (10 mL). The mixture was stirred at rt overnight then concentrated and carried over to the next step (200 mg, crude).

### Step 4:

Compound 4 (200 mg, 0.40 mmol) was dissolved in DCM (4 mL). TFA (2 mL) was added at rt and the mixture was stirred for 2 h. The reaction mixture was concentrated and purified by Prep-HPLC (acid method). White solid was afforded as Compound 006 (46 mg, TFA salt). LCMS: m/z=395.1 (M+H)⁺. ¹H NMR (400 MHz, MeOD) δ 8.65 (s, 2H), 7.80 (d, J=2.0 Hz, 1H), 7.37 (dd, J=8.3, 2.1 Hz, 1H), 7.28-7.23 (m, 2H), 7.05 (dd, J=5.1, 3.6 Hz, 1H), 6.95 (d, J=8.3 Hz, 1H), 3.67 (s, 4H), 3.14 (d, J=4.7 Hz, 4H), 2.74 (s, 3H).

### Example 6

### Synthesis of Compound 007

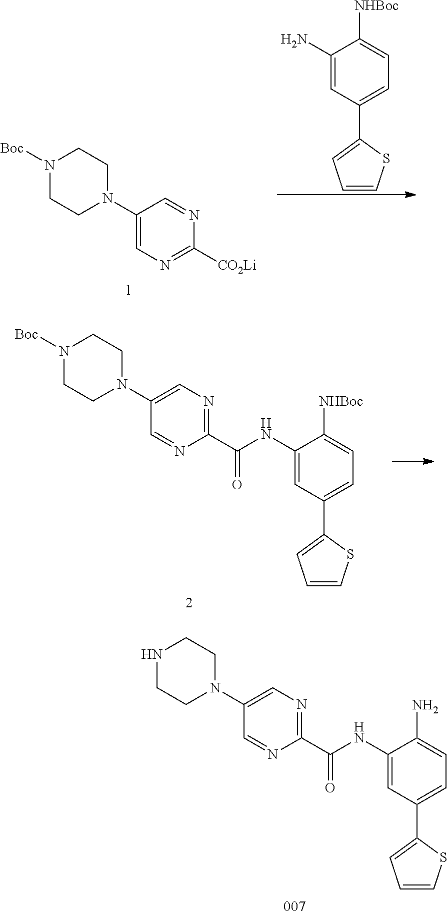

### Step 1:

Compound 1 (30 mg, 0.10 mmol) and HATU (68 mg, 0.18 mmol) were combined in DMF (2 mL) and stirred at rt for 15 min. tert-Butyl-2-amino-4-(thiophen-2-yl)phenylcarbamate (28 mg, 0.10 mmol) was added and the reaction mixture was stirred at rt for 3 h. The reaction was diluted H₂O (5 mL) and Compound 2 precipitated out as a white solid (54 mg, 93%).

### Step 2:

To a solution of compound 2 (54 mg, 0.10 mmol) in DCM (2 mL) at 0° C. was added TFA (1 mL) dropwise. The reaction mixture was stirred at rt for 1 h then concentrated in vacuo. The resulting yellow solid was rinsed with ether, dissolved into water (10 mL), re-crystallized from water, and dried by lyophilization. Compound 007 was isolated as a yellow solid (43 mg, TFA salt). LCMS: m/z=381.1 (M+H)⁺. ¹H NMR (400 MHz, DMSO) δ 9.95 (s, 1H), 8.63 (s, 2H), 7.78 (d, J=2.0 Hz, 1H), 7.38 (dd, J=5.1, 1.0 Hz, 1H), 7.31-7.19 (m, 2H), 7.06 (dd, J=5.1, 3.6 Hz, 1H), 6.85 (d, J=8.3 Hz, 1H), 5.14 (s, 2H), 3.49 (s, 4H), 3.00 (s, 4H).

### Example 7

### Synthesis of Compound 009

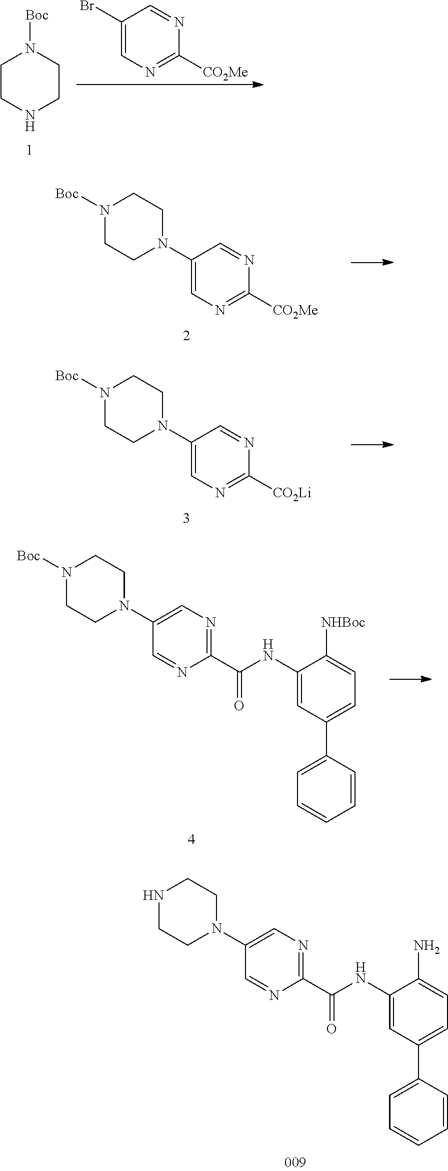

### Step 1:

Under nitrogen, compound 1 (275 mg, 1.48 mmol), methyl-5-bromopyrimidine-2-carboxylate (165 mg, 0.76 mmol), Ruphos (33 mg, 0.07 mmol), Pd₂(dba)₃ (17 mg, 0.018 mmol) and Cs₂CO₃ (748 mg, 2.29 mmol) were combined in toluene (8 mL) and heated to 100° C. overnight. The crude residue was purified by preparative TLC (silica gel, GF254 10-40u, 25*25cm) with petroleum ether/EtOAc (1:1) to afford Compound 2 as a white solid (80 mg, 40%).

### Step 2:

Compound 2 (80 mg, 0.25 mmol) and LiOH (10 mg, 0.25 mmol) were combined in MeOH (0.3 mL), THF (2 mL) and H₂O (1 mL) and was stirred at 0° C. for 3 h. The reaction mixture was concentrated in vacuo to afford Compound 3 as a white solid (30 mg, 40%).

### Step 3:

Compound 3 (30 mg, 0.10 mmol) and HATU (68 mg, 0.18 mmol) were dissolved in DMF (2 mL) and stirred at rt for 15 min. tert-Butyl 3-aminobiphenyl-4-ylcarbamate (28 mg, 0.10 mmol) was added and the reaction was stirred at rt for 3 h. The crude mixture was diluted with H₂O (5 mL), and the solid that precipitated was filtered to afford Compound 4 as a yellow solid (50 mg, 88%).

### Step 4:

To a solution of compound 4 (50 mg, 0.087 mmol) in DCM (2 mL) at 0° C. was added TFA (1 mL) dropwise. The reaction was stirred at rt for 2 h then concentrated in vacuo. The crude solid rinsed with ether then added to H₂O (50 mL). The yellow solid was re-crystallized from water and dried by lyophilization to afford Compound 009 as a yellow solid (47 mg, TFA salt). LCMS: m/z=375.2 (M+H)^{+ 1}H NMR (400 MHz, DMSO) δ 10.03 (s, 1H), 9.20 (s, 2H), 8.70 (s, 2H), 7.81 (s, 1H), 7.57 (d, J=7.6 Hz, 2H), 7.41 (t, J=7.5 Hz, 2H), 7.37-7.20 (m, 2H), 6.94 (d, J=8.2 Hz, 1H), 3.70 (s, 4H), 3.28 (s, 4H).

### Example 8

### Synthesis of Compound 010

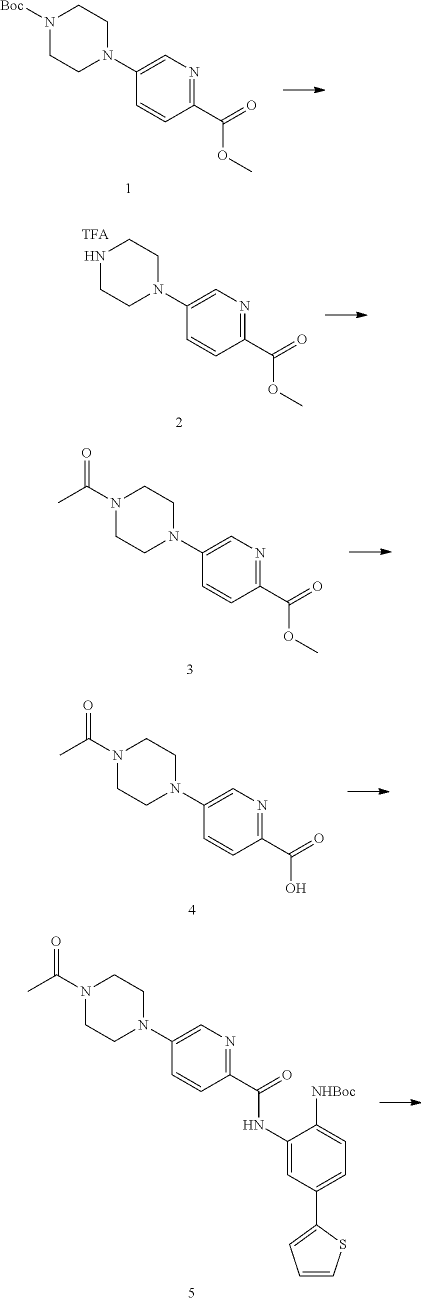

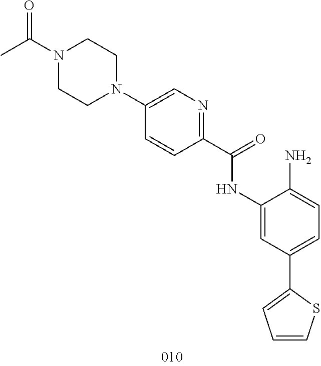 [SMILES: CC(=O)N1CCN(c2ccc(C(=O)Nc3cc(-c4cccs4)ccc3N)nc2)CC1]

### Step 1:

To a solution of compound 1 (500 mg, 1.56 mmol) in DCM (10 mL) was added TFA (2 mL). The reaction mixture was stirred at rt for 2 h. After the reaction was completed, the mixture was concentrated and rinsed with ether. The resulting solid was filtered to afford Compound 2 as a white solid (500 mg, crude).

### Step 2:

To a solution of compound 2 (500 mg, crude) in DCM (10 mL) was added acetyl chloride (1.5 eq) and DIPEA (3.0 eq). The reaction mixture was stirred at rt for 2 h. After the reaction had completed, the mixture was extracted with DCM/water and concentrated to afford Compound 3 as a yellow solid (430 mg, crude).

### Step 3:

To a solution of compound 3 (430 mg, crude) in MeOH and THF (10 MI, v/v=1) was added NaOH (1.5 eq, 2N). The mixture was stirred at 60° C. for 2 h. After the reaction had completed, the mixture was concentrated to afford Compound 4 as a yellow solid (371 mg, crude).

### Step 4:

To a solution of compound 4 (371 mg, crude) in Py (8 mL) was added EDCI (2.0 eq) and tert-butyl 2-amino-4-(thiophen-2-yl) phenylcarbamate (1.0 eq). The mixture was stirred at rt overnight. After the reaction was complete, the crude material was purified via column chromatography to afford Compound 5 as a white solid (226 mg, 28%, 4 steps)

### Step 5:

To a solution of compound 5 (226 mg, 0.43 mmol) in DCM (5 mL) at 0° C. was added TFA (1 mL) dropwise. The reaction was stirred at rt for 2 h and concentrated in vacuo. The crude material was neutralized with saturated NaHCO₃, and the resulting solid was rinsed with ether to afford Compound 010 as a yellow solid (118 mg, 65%). LCMS: m/z=422 (M+H)⁺ ¹ H NMR (400 MHz, DMSO) δ 9.88 (s, 1H), 8.41 (s, 1H), 8.10-7.74 (m, 2H), 7.61-6.70 (m, 6H), 5.10 (s, 2H), 3.62 (s, 4H), 3.50-3.35 (m, 4H), 2.06 (s, 3H).

### Example 9

### Synthesis of Compound 011

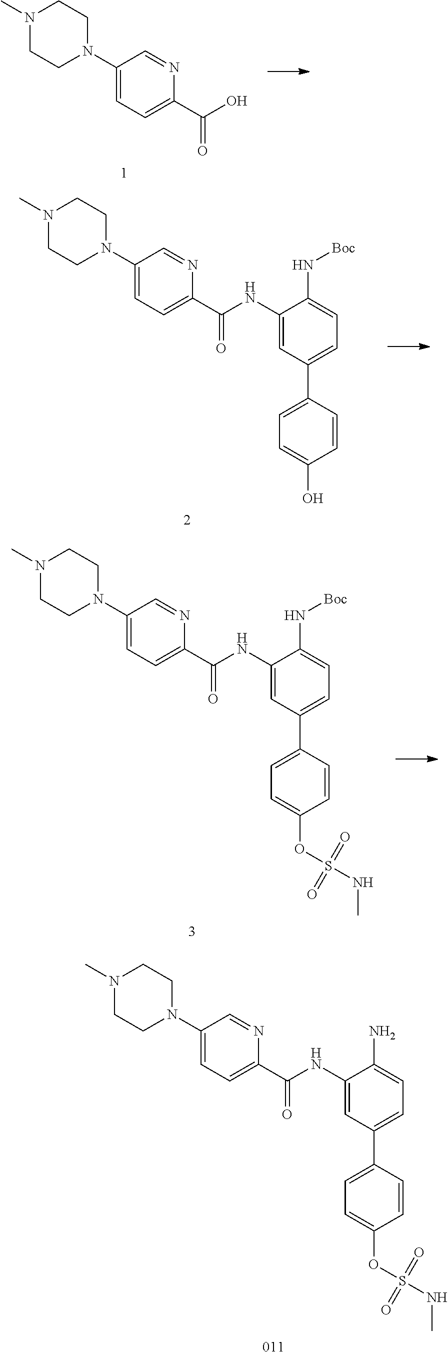

### Step 1:

Compound 1 (221 mg, 1 mmol), tert-butyl 3-amino-4-hydroxybiphenyl-4-ylcarbamate (300 mg, 1 mmol), and EDCI (382 mg, 2 mmol) were combined in Py (5 mL), and the reaction was stirred at rt overnight. The reaction mixture was concentrated and purified via column chromatography on silica gel to afford compound 2 (250 mg, 50%) as yellow solid.

### Step 2:

To a solution of compound 2 (250 mg, 0.5 mmol) in DMA (5 mL) was added NaH (60% in mineral oil, 80 mg, 2 mmol) and methylsulfamoyl chloride (129 mg, 1 mmol). The reaction mixture was stirred at 0° C. for 3 h, then diluted with water (30 mL), and filtered to afford compound 3 (209 mg, 70%) as yellow solid.

### Step 3:

Compound 3 (298 mg, 0.5 mmol) and TFA (2 mL) were combined in DCM (5 mL) and stirred at rt for 1 h. The reaction mixture was concentrated to Compound 011 (198 mg, 80%) as yellow solid. LCMS: m/z=497 (M+H)^{+ 1}H NMR (400 MHz, DMSO) δ 9.89 (s, 1H), 8.39 (d, J=2.7 Hz, 1H), 7.96 (d, J=8.8 Hz, 1H), 7.88 (d, J=2.0 Hz, 1H), 7.63 (d, J=8.7 Hz, 2H), 7.48 (dd, J=8.9, 2.8 Hz, 1H), 7.35-7.21 (m, 3H), 6.91 (d, J=8.3 Hz, 1H), 5.08 (s, 2H), 3.44-3.35 (m, 4H), 2.73 (s, 3H), 2.46 (d, J=4.9 Hz, 4H), 2.24 (s, 3H), 1.78 (s, 2H).

### Example 10

### Synthesis of Compound 012

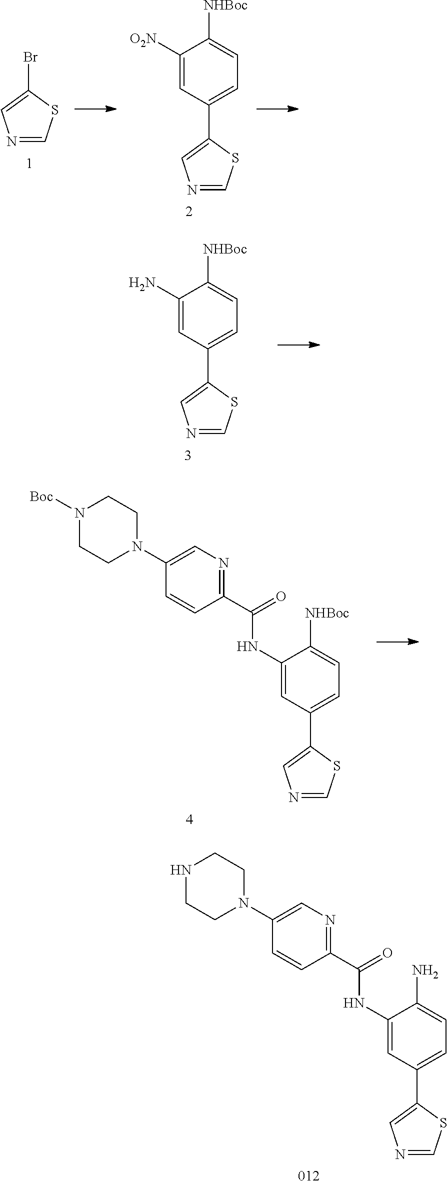 [SMILES: Brc1cncs1 ; CC(C)(C)OC(=O)Nc1ccc(-c2cncs2)cc1[N+](=O)[O-] ; CC(C)(C)OC(=O)Nc1ccc(-c2cncs2)cc1N ; CC(C)(C)OC(=O)Nc1ccc(-c2cncs2)cc1NC(=O)c1ccc(N2CCN(C(=O)OC(C)(C)C)CC2)cn1 ; Nc1ccc(-c2cncs2)cc1NC(=O)c1ccc(N2CCNCC2)cn1]

### Step 1:

Compound 1 (546 mg, 3.3 mmol), tert-butyl-2-nitro-4-(4,4,5,5-tetramethyl-1,3,2-dioxaborolan-2-yl) phenylcarbamate (1092 mg, 3 mmol), Pd(dppf)Cl₂(123 mg, 0.15 mmol) and K₂CO₃ (828 mg, 6 mmol) were added to 1,4-dioxane (10 mL) and H₂O (3 mL). The mixture was stirred at 90° C. overnight then purified by CombiFlash (PE/EA=2/1) to afford Compound 2 (240 mg, 50%) as a yellow solid.

### Step 2:

Compound 2 (240 mg, 0.75 mmol), FeCl₃ (13 mg, 0.08 mmol), activated carbon (30 mg) were added into EtOH (10 ml). The mixture was heated to 60° C. and N₂H₄H₂O (2 mL) was added dropwise. The reaction was stirred for 3 h, then filtered, concentrated, and rinsed with Et₂O. Compound 3 was afforded as a white solid (210 mg, 96%).

### Step 3:

Compound 3 (62 mg, 0.21 mmol), 5-(4-(tert-butoxycarbonyl) piperazin-1-yl) picolinic acid (64 mg, 0.21 mmol) and EDCI (121 mg, 0.63 mmol) were added into Py (5 mL). The mixture was stirred at rt overnight then concentrated and purified by CombiFlash (PE/EA=1/1) to afford compound 4 (40 mg, 34%).

### Step 4:

Compound 4 (40 mg, 0.07 mmol) was dissolved in DCM (2 mL). TFA (1 mL) was added at rt, and the reaction was stirred for 2 h. The reaction was then concentrated and rinsed with Et₂O. Yellow solid was afforded as Compound 012 (46 mg, TFA salt). LCMS: m/z=381.2 (M+H)⁺. ¹H NMR (400 MHz, DMSO) δ 9.94 (s, 1H), 8.95 (s, 3H), 8.45 (d, J=2.6 Hz, 1H), 8.08 (s, 1H), 8.02 (d, J=8.8 Hz, 1H), 7.83 (d, J=2.0 Hz, 1H), 7.57 (dd, J=8.8, 2.8 Hz, 1H), 7.32 (dd, J=8.3, 2.0 Hz, 1H), 6.90 (d, J=8.3 Hz, 1H), 3.61 (d, J=5.2 Hz, 4H), 3.28 (s, 4H).

### Example 11

### Synthesis of Compound 013

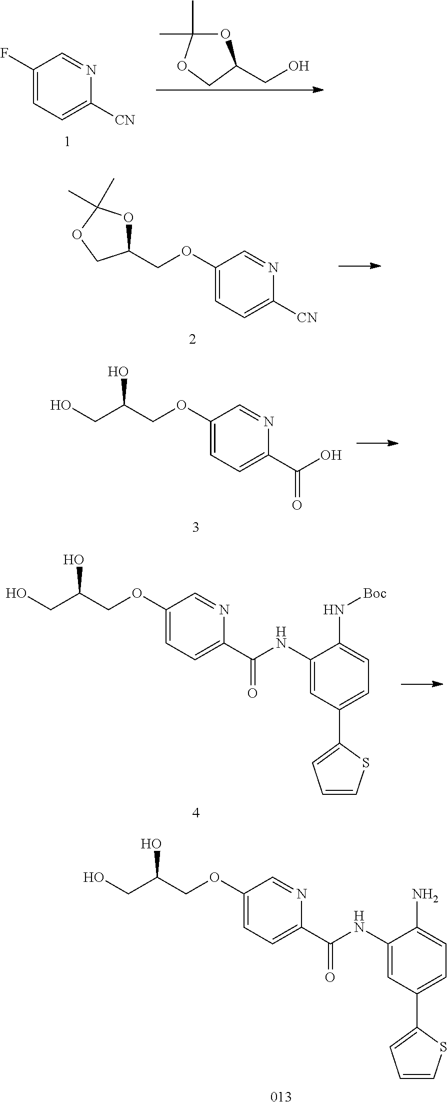

### Step 1:

To a solution of 5-fluoropicolinonitrile (366 mg, 3 mmol) and (S)-(2,2-dimethyl-1,3-dioxolan-4-yl)methanol (528 mg, 4 mmol) in THF (15 mL) was added NaH (60% in mineral oil, 160 mg, 4 mmol) at 0° C. The reaction was heated to 60° C. and stirred for 4 h. The reaction mixture was diluted with water (50 mL) and filtered to afford Compound 2 (700 mg) without purification.

### Step 2:

Compound 2 (700 mg, crude) was added to HCl (37%, 10 mL) and stirred at 80° C. overnight. The mixture was concentrated. The crude material was rinsed with EA (20 mL) and filtered to afford Compound 3 (400 mg, 57% for two steps) as yellow solid.

### Step 3:

Compound 3 (213 mg, 1 mmol), tert-butyl-2-amino-4-(thiophen-2-yl)phenylcarbamate (290 mg, 1 mmol), and EDCI (382 mg, 2 mmol) were dissolved in Py (5 mL) and stirred at rt overnight. The mixture was concentrated and purified via column chromatography on silica gel to afford compound 4 (400 mg, 70%) as yellow solid.

### Step 4:

Compound 4 (485 mg, 1 mmol) and TFA (2 mL) were combined in DCM (5 mL) and stirred at rt for 1 h. The mixture was concentrated to get compound 013 (250 mg, 65%) as yellow solid. LCMS: m/z=386 (M+H)^{+ 1}H NMR (400 MHz, DMSO) δ 9.97 (s, 1H), 8.41 (d, J=2.5 Hz, 1H), 8.11 (d, J=8.7 Hz, 1H), 7.82 (s, 1H), 7.62 (dd, J=8.7, 2.7 Hz, 1H), 7.37 (d, J=4.8 Hz, 1H), 7.27 (dd, J=11.4, 2.7 Hz, 2H), 7.11-6.96 (m, 1H), 6.85 (d, J=8.3 Hz, 1H), 5.21-5.02 (m, 3H), 4.76 (t, J=5.6 Hz, 1H), 4.21 (dd, J=10.1, 3.6 Hz, 1H), 4.07 (dd, J=10.1, 6.2 Hz, 1H), 3.85 (dd, J=9.6, 5.3 Hz, 1H), 3.48 (t, J=5.5 Hz, 2H).

### Example 12

### Synthesis of Compound 014

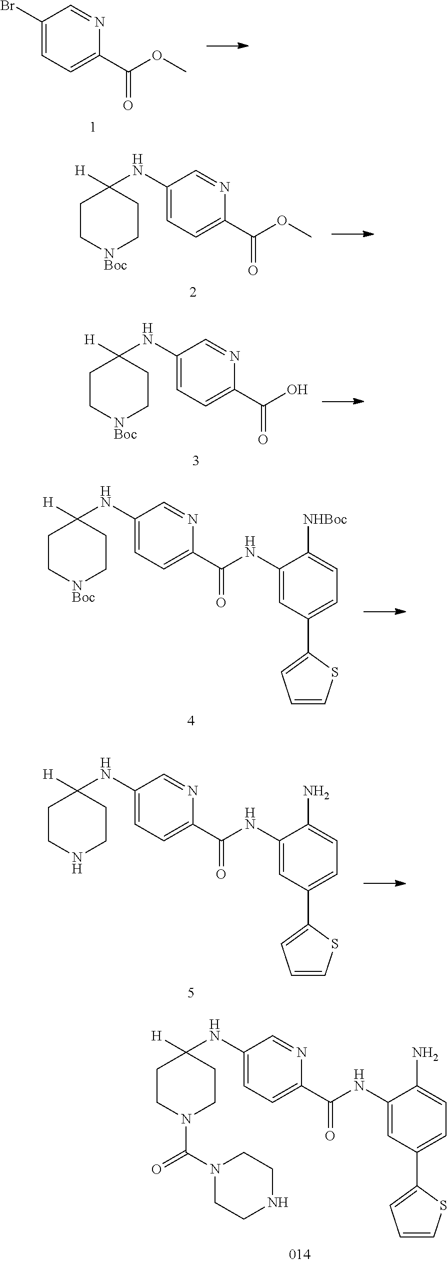 [SMILES: COC(=O)c1ccc(Br)cn1 ; COC(=O)c1ccc(NC2CCN(C(=O)OC(C)(C)C)CC2)cn1 ; CC(C)(C)OC(=O)N1CCC(Nc2ccc(C(=O)O)nc2)CC1 ; CC(C)(C)OC(=O)Nc1ccc(-c2cccs2)cc1NC(=O)c1ccc(NC2CCN(C(=O)OC(C)(C)C)CC2)cn1 ; Nc1ccc(-c2cccs2)cc1NC(=O)c1ccc(NC2CCNCC2)cn1 ; Nc1ccc(-c2cccs2)cc1NC(=O)c1ccc(NC2CCN(C(=O)N3CCNCC3)CC2)cn1]

### Step 1:

Methyl-5-bromopicolinate (3.35 g, 15.50 mmol), tert-butyl-4-aminopiperidine-1-carboxylate (3.1 g, 15.48 mmol), tris(dibenzylideneacetone)dipalladium (341 mg, 0.37 mmol), RuPhos (600 mg, 0.66 mmol) and cesium carbonate (10.08 g, 30.92 mmol) were combined in toluene (100 mL) and heated at 100° C. for 18 h. The reaction was allowed to cool rt and filtered. The filtrate was concentrated in vacuo, and the crude residue was rinsed with PE/EA to give compound 2 as a light yellow solid (1.22 g, yield: 23%).

### Step 2:

Compound 2 (1.22 g, 3.62 mmol) and LiOH (456 mg, 10.86 mmol) were combined in THF (54 mL), water (18 mL) and MeOH (9 mL) and stirred at 50° C. until the reaction was completed. The crude mixture was concentrated in vacuo, and the aqueous phase was neutralized to pH 6 by addition 2M HCl at 0° C. The precipitate was collected and dried in vacuo to give compound 3 as a light yellow solid (735 mg, yield: 63%).

### Step 3:

Compound 3 (645 mg, 2.04 mmol), tert-butyl 2-amino-4-(thiophen-2-yl)phenylcarbamate (590 mg, 2.04 mmol) and EDCI (780 mg, 4.07 mmol) were dissolved in pyridine (20 mL) and stirred at rt for 18 h. The reaction mixture was concentrated in vacuo and the crude material was washed with water and dried in vacuo to give crude Compound 4 (1.40 g).

### Steps 4 and 5:

At 0° C., to a solution of compound 4 (1.4 g) in DCM (10 mL) was added TFA (10 mL). It was stirred at 0° C. for 2 h then concentrated in vacuo. The crude residue was neutralized at 0° C. with aqueous saturated NaHCO₃ to near pH 8. The precipitate was collected and dried in vacuo to give N-(2-amino-5-(thiophen-2-yl)phenyl)-5-(piperidin-4-ylamino)picolinamide (946 mg). The above product (200 mg, 0.51 mmol), tert-butyl-4-(chlorocarbonyl)piperazine-1-carboxylate (126 mg, 0.51 mmol) and TEA (70.6 uL, 0.51 mmol) in DMF (6 mL) was stirred at rt for 18 h. The crude material was purified by prep-HPLC to give Compound 014 as a white solid (122 mg). LCMS: m/z=506.2 (M+H)⁺. ¹H NMR (400 MHz, DMSO) δ 9.77 (s, 1H), 8.07 (d, J=2.4 Hz, 1H), 7.89 (s, 1H), 7.87 (d, J=7.6 Hz, 1H), 7.37 (dd, J=5.0, 1.0 Hz, 1H), 7.25-7.23 (m, 2H), 7.10 (dd, J=8.6, 2.6 Hz, 1H), 7.05 (dd, J=5.0, 3.4 Hz, 1H), 6.85 (d, J=8.4 Hz, 1H), 6.61 (d, J=7.6 Hz, 1H), 5.06 (s, 2H), 3.56 (d, J=12.0 Hz, 3H), 3.07 (s, 4H), 2.66 (brs, 3H), 1.92 (m, 2H), 1.36 (m, 2H).

### Example 13

### Synthesis of Compound 015

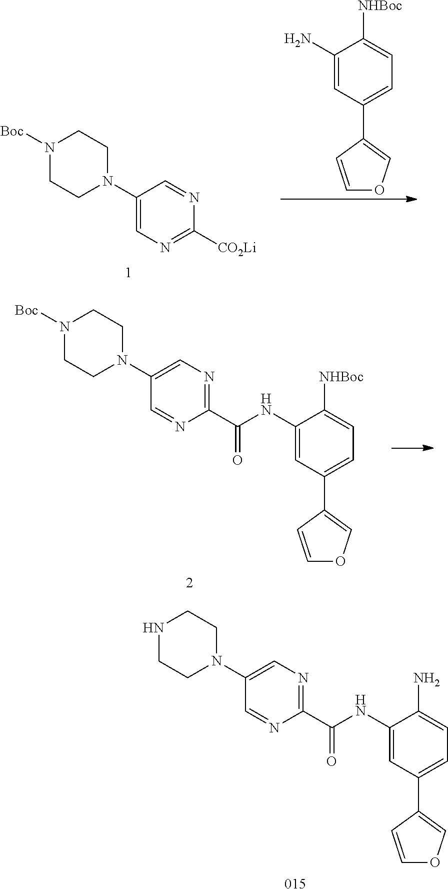

### Step 1:

Compound 1 (160 mg, 0.53 mmol, lithium salt) and HATU (225 mg, 0.60 mmol) were combined in DMF (5 mL) and the reaction was stirred at rt for 15 min. tert-Butyl-2-amino-4-(furan-3-yl)phenylcarbamate (90 mg, 0.33 mmol) was added. The mixture was stirred at rt for 4 h then diluted with water (10 mL). The resulting precipitate was filtered to afford Compound 2 as a yellow solid (180 mg, 96%).

### Step 2:

To a solution of compound 2 (180 mg, 0.32 mmol) in DCM (2 ml) at 0° C. was added TFA (2 ml) drop wise. The reaction mixture was stirred at rt for 1 h then concentrated in vacuo. The crude residue was rinsed with ether then washed with water (50 mL). The red solid was re-crystallized from water and dried by lyophilization to afford a red solid Compound 015 (149 mg, TFA salt). LCMS: m/z=365.2 (M+H)^{+ 1}H NMR (400 MHz, DMSO) δ 10.02 (s, 1H), 9.18 (s, 2H), 8.69 (s, 2H), 7.99 (s, 1H), 7.68 (d, J=7.4 Hz, 2H), 7.27 (d, J=8.1 Hz, 1H), 6.97-6.64 (m, 2H), 3.69 (s, 4H), 3.29 (s, 4H).

### Example 14

### Synthesis of Compound 016

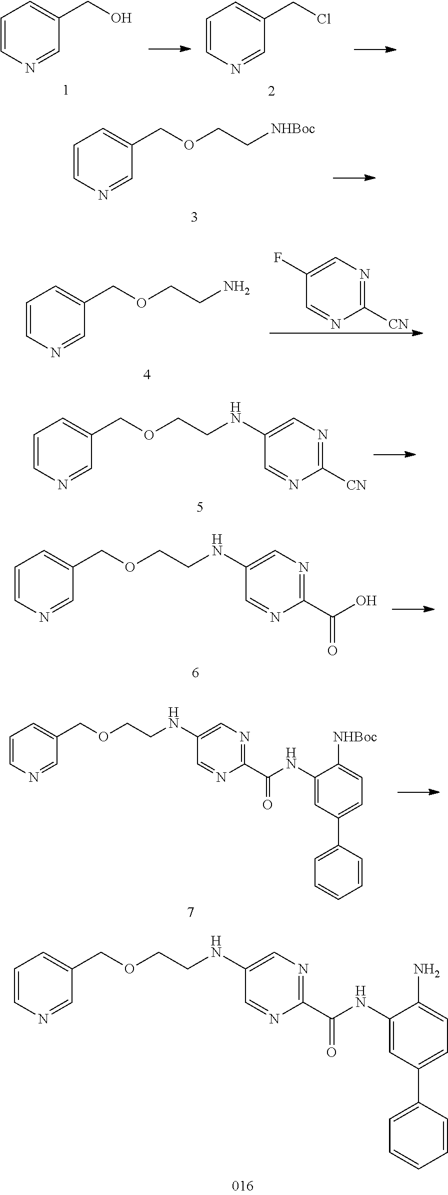

### Step 1:

To a solution of pyridin-3-ylmethanol (6.55 g, 60.04 mmol) in DCM (20 mL) was added SOCl₂ (7.3 mL, 100.86 mmol) at 0° C. The reaction was allowed to warm to rt and stirred for 15 min. The reaction was then heated to 60° C. and stirred for 1 h, then concentrated in vacuo. The crude residue was used in next step.

### Step 2:

To a mixture of tert-butyl 2-hydroxyethylcarbamate (2.75 g, 17.07 mmol) and KOH (1.92 g, 34.13 mmol) in DMSO (20 mL) was added 3-(chloromethyl)pyridine hydrochloride (2.00 g, 12.19 mmol). The reaction was stirred at rt for 6 h then diluted with water at 0° C. The resulting mixture was extracted with EA. The combined organic layers were washed with water and concentrated in vacuo. The crude material was purified via column chromatography (PE/EA=2/1 to 1/1) to give Compound 3 as the colorless oil (2.22 g, yield: 72%).

### Step 3:

A mixture of tert-butyl 2-(pyridin-3-ylmethoxy)ethylcarbamate (2.22 g, 8.80 mmol) and concentrated HCl (5 mL) in water-MeOH (15 mL) was stirred 50° C. for 2 h. The reaction mixture was concentrated in vacuo, and the residue was adjusted to pH 7 by addition of aqueous NaOH. The mixture was extracted with EA. The combined EA layers were concentrated in vacuo to give crude Compound 4 as the colorless oil (700 mg, yield: 52%).

### Step 4:

Under N₂, 2-(pyridin-3-ylmethoxy)ethanamine (380 mg, 2.50 mmol), 5-fluoropyrimidine-2-carbonitrile (369 mg, 3.00 mmol) and DIEA (1.24 mL, 7.49 mmol) was heated to 150° C. for 1 h. The reaction was cooled to rt and diluted with water. The resulting mixture was extracted with EA. The combined EA layers were concentrated in vacuo, and the crude material was purified by prep-TLC to give compound 5 as a brown solid (547 mg, yield: 82%).

### Step 5:

Under N₂, a mixture of 5-(2-(pyridin-3-ylmethoxy)ethylamino)pyrimidine-2-carbonitrile (527 mg, 2.06 mmol) and NaOH (248 mg, 6.19 mmol) in MeOH (13 mL) and water (13 mL) was heated to 100° C. for 18 h. The reaction was cooled to rt and MeOH was removed in vacuo. The crude aqueous material was adjusted to pH 7 then concentrated in vacuo to give crude compound 6 as a light brown solid (1.082 g, with NaCl).

### Step 6:

A mixture of 5-(2-(pyridin-3-ylmethoxy)ethylamino)pyrimidine-2-carboxylic acid (300 mg, 0.59 mmol), tert-butyl-3-aminobiphenyl-4-ylcarbamate (168 mg, 0.59 mmol) and EDCI (226 mg, 1.18 mmol) in pyridine (18 mL) was stirred at rt for 18 h. The reaction was then concentrated in vacuo and the crude product was used in next step without purification.

### Step 7:

To a mixture of crude tert-butyl-3-(5-(2-(pyridin-3-ylmethoxy)ethylamino)-pyrimidine-2-carboxamido)biphenyl-4-ylcarbamate (crude, 0.66 mmol) in DCM (2 mL) was added TFA (2 mL) at 0° C. The reaction was allowed to warm to rt and stirred for 2 h. The crude material was concentrated in vacuo and purified by prep-HPLC to give Compound 016 as a yellow solid (53.6 mg, yield: 19%, lots SP-0018434-062). LCMS: m/z=441.1 (M+H)⁺. 1H NMR (400 MHz, DMSO) δ 9.87 (s, 1H), 8.56 (d, J=1.8 Hz, 1H), 8.50 (dd, J=4.7, 1.4 Hz, 1H), 8.32 (s, 2H), 7.82 (d, J=2.0 Hz, 1H), 7.76 (d, J=7.8 Hz, 1H), 7.56 (d, J=7.5 Hz, 2H), 7.44-7.34 (m, 3H), 7.31-7.20 (m, 2H), 6.90 (t, J=6.0 Hz, 2H), 5.05 (s, 2H), 4.59 (s, 2H), 3.67 (t, J=5.2 Hz, 2H), 3.48-3.43 (m, 2H).

### Example 15

### Synthesis of Compound 017

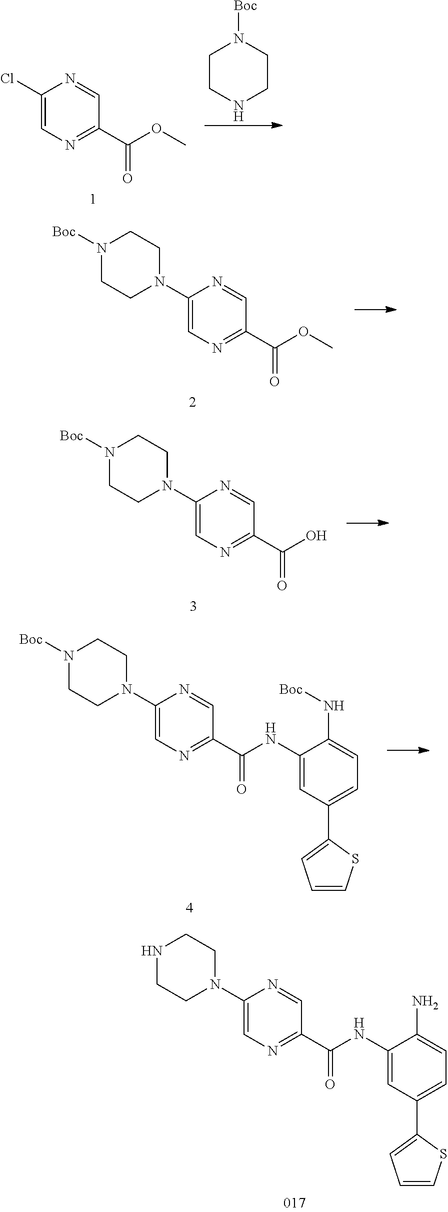 [SMILES: COC(=O)c1cnc(Cl)cn1 ; CC(C)(C)OC(=O)N1CCNCC1 ; COC(=O)c1cnc(N2CCN(C(=O)OC(C)(C)C)CC2)cn1 ; CC(C)(C)OC(=O)N1CCN(c2cnc(C(=O)O)cn2)CC1 ; CC(C)(C)OC(=O)Nc1ccc(-c2cccs2)cc1NC(=O)c1cnc(N2CCN(C(=O)OC(C)(C)C)CC2)cn1 ; Nc1ccc(-c2cccs2)cc1NC(=O)c1cnc(N2CCNCC2)cn1]

### Step 1:

tert-Butyl piperazine-1-carboxylate (3.72 g, 20 mmol), methyl 5-chloropyrazine-2-carboxylate (1.72 g, 10 mmol) and DIPEA (2.02 g, 20 mmol) were combined in 1,4-dioxane (15 mL). The mixture was stirred overnight at 100° C. When the reaction was finished, it was diluted with EA and washed with brine. The crude material was purified by flash column chromatography (EA, 100%). Solid 2 was isolated (6 g, impure).

### Step 2:

Methyl 5-(4-(tert-butoxycarbonyl) piperazin-1-yl) pyrazine-2-carboxylate (2 g) was combined with aqueous NaOH (5 mL, 2M) in a solution of EtOH (5 mL)/THF (5 mL). The mixture was stirred at 60° C. for 2 h. When the reaction was finished, the pH was adjusted to 1, and the crude material was extracted with EA. Compound 3 was afforded as a pink solid (300 mg, 15% yield).

### Step 3:

5-(4-(tert-Butoxycarbonyl) piperazin-1-yl) pyrazine-2-carboxylic acid (200 mg, 0.65 mmol), tert-butyl 2-amino-4-(thiophen-2-yl) phenylcarbamate (157 mg, 0.54 mmol), and EDCI (311 mg, 1.62 mmol) were added into Py (5 mL). The mixture was stirred at rt overnight. When the reaction was complete, it was concentrated and washed with Et₂O. Compound 4 was isolated as a yellow solid (310 mg, 65% yield).

### Step 4:

tert-Butyl 4-(5-(2-(tert-butoxycarbonylamino)-5-(thiophen-2-yl) phenylcarbamoyl) pyrazin-2-yl) piperazine-1-carboxylate (300 mg, 0.52 mmol) was dissolved into DCM (2 mL). Then TFA (2 mL) was added. The mixture was stirred at rt for 2 h. When the reaction finished, it was adjusted to pH 10 and extracted with EA. The organic extract was concentrated and the resulting solid was washed with Et₂O to afford yellow solid Compound 017 (140 mg, 69% yield). LCMS: m/z=381.2 (M+H)⁺. ¹H NMR (400 MHz, DMSO) δ 9.68 (s, 1H), 8.72 (s, 1H), 8.34 (s, 1H), 7.79 (s, 1H), 7.37 (d, J=4.7 Hz, 1H), 7.26 (d, J=8.7 Hz, 2H), 7.06 (s, 1H), 6.84 (d, J=8.1 Hz, 1H), 5.10 (s, 2H), 3.68 (s, 4H), 2.83 (s, 4H).

### Example 16

### Pharmacokinetics

Male SD rats were fasted overnight. Compounds of the invention were dissolved in dimethyl acetamide at 10 times the final concentration, then Solutol HS 15 (BASF) was added to a final concentration of 10%. Finally 80% saline was added and vortexed to achieve a clear solution. For the IV dosing three animals were injected via the foot dorsal vein with 1 mg/kg compound. For the PO dosing 5 mg/kg of compound was delivered by oral gavage. Blood was collected via the tail vein into K2EDTA tubes at 5 minutes, 15 minutes, 30 minutes, 1 hour, 2 hours, 4 hours, 8 hours and 24 hours after dosing. The blood was centrifuged at 2000 g for 5 minutes at 4° C. to obtain plasma. The plasma was extracted with acetonitrile and the level of compound was analyzed by LC/MS/MS. The level of compound in plasma was calculated from a standard curve in rat plasma. The IV clearance (L/h/kg) and area under the curve (h*ng/mL) were calculated using WinNonLin software. The dose adjusted area under the curve for the IV and oral dosing were used to calculate the oral bioavailability.

Pharmacokinetic properties were assessed in a rat cassette dosing experiment. The IV clearance (IV Clr.) is in units of L/hr/kg. The oral maximum plasma concentration (PO Cmax) is in units of ng/ml. The oral plasma half-life (PO T1/2) is in units of hours. The oral area under the curve (PO AUC) is in units of hours*ng/ml. The fraction absorbed by the oral route (F %) is a percentage of the oral area under the curve to the IV area under the curve, dose adjusted. A summary of results is presented in Table 2 and Table 3, below.

TABLE 2

| | PK Parameters (Mouse IV) | | |
|---|---|---|---|
| | AUC(last) Plasma | AUC(last) Brain | Brain AUC/ |
| Compound | (ng × hr/mL) | (ng × hr/mL) | Plasma AUC |
| 001 | 1400-1500 | 19000-20000 | 10-15 |
| 002 | 400-500 | 9000-10000 | 20-25 |
| 003 | 1000-1100 | 3000-4000 | 1-5 |
| 004 | 700-800 | 500-600 | 0.1-1.5 |
| 005 | 600-700 | 600-700 | 0.8-1.2 |
| 006 | 200-300 | 700-800 | 2-4 |
| 007 | 400-500 | 30-40 | 0.01-0.1 |
| 008 | 600-700 | 200-300 | 0.1-1.5 |
| 009 | 300-400 | 300-400 | 0.8-1.5 |
| 010 | 500-600 | 200-300 | 0.1-1.5 |
| 011 | 200-300 | 100-200 | 0.1-1.5 |
| 012 | 800-900 | 400-500 | 0.1-1.5 |
| 013 | 1100-1200 | 200-300 | 0.1-0.3 |
| 014 | 1500-1600 | 100-200 | 0.05-0.1 |
| 015 | 500-600 | 100-200 | 0.1-1.5 |
| 016 | 300-400 | 0-100 | 0.01-0.1 |
| 017 | 600-700 | 9000-10000 | |

TABLE 3

| | PK Parameters (Mouse IP) | | |
|---|---|---|---|
| | AUC(last) Plasma | AUC(last) Brain | Brain AUC/ |
| Compound | (ng × hr/mL) | (ng × hr/mL) | Plasma AUC |
| 001 | 6700-6800 | 64700-64800 | 1-5 |
| 002 | 2400-2500 | 23100-23200 | 5-10 |
| 003 | 4700-4800 | 7900-8000 | 1-5 |
| 004 | 2300-2400 | 2100-2200 | 0.1-1.5 |
| 005 | 2300-2400 | 2000-2100 | 0.1-1.5 |
| 006 | 800-900 | 700-800 | 0.1-1.5 |
| 007 | 1800-1900 | 900-1000 | 0.1-1.5 |
| 008 | 2900-3000 | 1300-1400 | 0.1-1.5 |
| 009 | 1700-1800 | 200-300 | 0.1-1.5 |
| 010 | 2000-2100 | 200-300 | 0.1-1.5 |
| 011 | 900-1000 | 0-100 | 0.1-1.5 |
| 012 | 4300-4400 | 300-400 | 0.01-0.1 |
| 013 | 5900-6000 | 300-400 | 0.01-0.1 |
| 014 | 7400-7500 | 200-300 | 0.01-0.1 |
| 015 | 3200-3300 | 0-100 | 0.01-0.1 |
| 016 | 900-1000 | 0-100 | 0.001-0.01 |
| 017 | 2700-2800 | 25000-25300 | |

### Example 17

### HDAC Enzyme Assays

Compounds for testing were diluted in DMSO to 50 fold the final concentration and a ten-point three-fold dilution series was made. The compounds were diluted in assay buffer (50 mM HEPES, pH 7.4, 100 mM KCl, 0.001% Tween-20, 0.05% BSA, 20 μM TCEP) to 6-fold their final concentration. The HDAC enzymes (purchased from BPS Biosciences) were diluted to 1.5-fold their final concentration in assay buffer. The tripeptide substrate and trypsin at 0.05 μM final concentration were diluted in assay buffer at 6-fold their final concentration. The final enzyme concentrations used in these assays were 3.3 ng/ml (HDAC1), 0.2 ng/ml (HDAC2) and 0.08 ng/ml (HDAC3). The final substrate concentrations used were 16 μM (HDAC1), 10 μM (HDAC2) and 17 μM (HDAC3).

Five μl of compounds and 20 μl of enzyme were added to wells of a black, opaque 384 well plate in duplicate. Enzyme and compound were incubated together at room temperature for 10 minutes. Five μl of substrate was added to each well, the plate was shaken for 60 seconds and placed into a Victor 2 microtiter plate reader. The development of fluorescence was monitored for 60 min and the linear rate of the reaction was calculated. The IC₅₀ was determined using Graph Pad Prism by a four parameter curve fit.

TABLE 4

| | IC₅₀ (nM) | | |
|---|---|---|---|
| Compound | HDAC1 | HDAC2 | HDAC3 |
| 001 | 10-20 | 30-40 | 480-490 |
| 002 | 1-10 | 10-20 | 200-210 |
| 003 | 1-10 | 20-30 | 170-180 |
| 004 | 1-10 | 50-60 | 440-450 |
| 005 | 1-10 | 10-20 | 110-120 |
| 006 | 1-10 | 40-50 | 80-90 |
| 007 | 10-20 | 90-100 | 170-180 |
| 008 | 1-10 | 20-30 | 260-270 |
| 009 | 10-20 | 70-80 | 200-210 |
| 010 | 1-10 | 10-20 | 120-130 |
| 011 | 1-10 | 10-20 | >2000 |
| 012 | 1-10 | 10-20 | 20-30 |
| 013 | 1-10 | 20-30 | 140-150 |
| 014 | 1-10 | 10-20 | 50-60 |
| 015 | 10-20 | 30-40 | 110-120 |
| 016 | 1-10 | 30-40 | 230-240 |
| 017 | 1-10 | 5-15 | 30-40 |

1. A compound of Formula I: 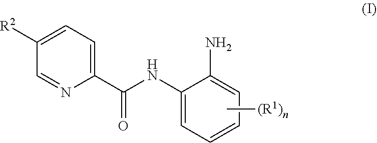
or a pharmaceutical acceptable salt thereof;
wherein:
R¹ is selected from the group consisting of aryl, halo, and heteroaryl, wherein aryl is optionally substituted with R³;
R² is selected from the group consisting of heterocycloalkyl, OR⁴, and N(H)R⁴, wherein heterocycloalkyl is optionally substituted with R⁴;
R³ is C₁-C₄ alkyl or —OSO₂N(H)C₁-C₄ alkyl;
R⁴ is independently, at each occurrence, selected from the group consisting of C₁-C₄ alkyl, —(C₁-C₄ alkyl)-O—(C₁-C₄ alkyl), —C(O)—C₁-C₄ alkyl, and -heterocycloalkyl-C(O)-heterocycloalkyl, wherein C₁-C₄ alkyl is optionally substituted with —OH; and
n is 1 or 2.

2. The compound of claim 1, wherein:
R¹ is selected from the group consisting of halo, phenyl, thiazole, and thienyl, wherein phenyl is optionally substituted with R³;
R² is selected from the group consisting of heterocycloalkyl, OR⁴, and N(H)R⁴, wherein heterocycloalkyl is optionally substituted with R⁴;
R³ is C₁-C₄ alkyl or —OSO₂N(H)C₁-C₄ alkyl;
R⁴ is independently, at each occurrence, selected from the group consisting of C₁-C₄ alkyl, —(C₁-C₄ alkyl)-O—(C₁-C₄ alkyl), —C(O)—C₁-C₄ alkyl, and -heterocycloalkyl-C(O)-heterocycloalkyl, wherein C₁-C₄ alkyl is optionally substituted with —OH; and
n is 1 or 2.

3. The compound of claim 1 or 2, wherein:
R¹ is selected from the group consisting of halo, phenyl, thienyl, and thiazole, wherein phenyl is optionally substituted with R³;
R² is selected from the group consisting of piperazine, OR⁴, and N(H)R⁴, wherein piperazine is optionally substituted with R⁴;
R³ is methyl or —OSO₂N(H)Me;
R⁴ is independently, at each occurrence, selected from the group consisting of C₁-C₃ alkyl, —(C₁-C₂ alkyl)-O-methyl, —C(O)-methyl, and -piperadine-C(O)-piperazine, wherein C₁-C₃ alkyl is optionally substituted with —OH; and
n is 1 or 2.

4. A compound of Formula II: 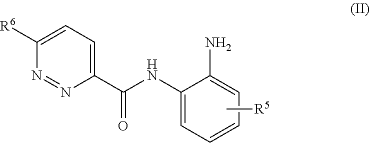
or a pharmaceutical acceptable salt thereof;
wherein:
R⁵ is aryl or heteroaryl; and
R⁶ is heterocycloalkyl or O—(C₁-C₄ alkyl)-O—(C₁-C₄ alkyl).

5. The compound of claim 4 wherein:
R⁵ is phenyl or thienyl; and
R⁶ is piperazine or O—(C₁-C₂ alkyl)-O-methyl.

6. A compound of Formula III: 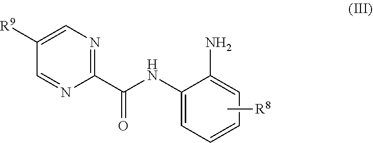
or a pharmaceutically acceptable salt thereof;
wherein:
R⁸ is aryl or heteroaryl;
R⁹ is N(H)R¹⁰ or heterocycloalkyl, wherein heterocycloalkyl is optionally substituted with C₁-C₄ alkyl; and
R¹⁰ is —(C₁-C₄ alkyl)-O—(C₁-C₄ alkyl), wherein C₁-C₄ alkyl is optionally substituted with heteroaryl.

7. The compound of claim 6, wherein:
R⁸ is selected from the group consisting of furanyl, phenyl, and thienyl;
R⁹ is N(H)R¹⁰ or heterocycloalkyl, wherein heterocycloalkyl is optionally substituted with C₁-C₄ alkyl; and
R¹⁰ is —(C₁-C₄ alkyl)-O—(C₁-C₄ alkyl), wherein C₁-C₄ alkyl is optionally substituted with heteroaryl.

8. The compound of claim 6 or 7, wherein:
R⁸ is selected from the group consisting of furanyl, phenyl, and thienyl;
R⁹ is N(H)R¹⁰ or piperazine, wherein piperazine is optionally substituted with methyl; and
R¹⁰ is —(C₁-C₂alkyl)-O—(C₁-C₂alkyl), wherein C₁-C₂ alkyl is optionally substituted with pyridine.

9. A compound of Formula IV: 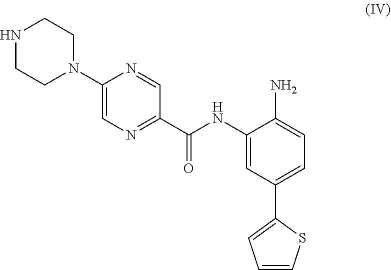 [SMILES: Nc1ccc(-c2cccs2)cc1NC(=O)c1cnc(N2CCNCC2)cn1]
or a pharmaceutically acceptable salt thereof.

10. The compound of any of claims 1-9, wherein the compound of Formula I, Formula II, Formula III, or Formula IV is selected from the group consisting of: 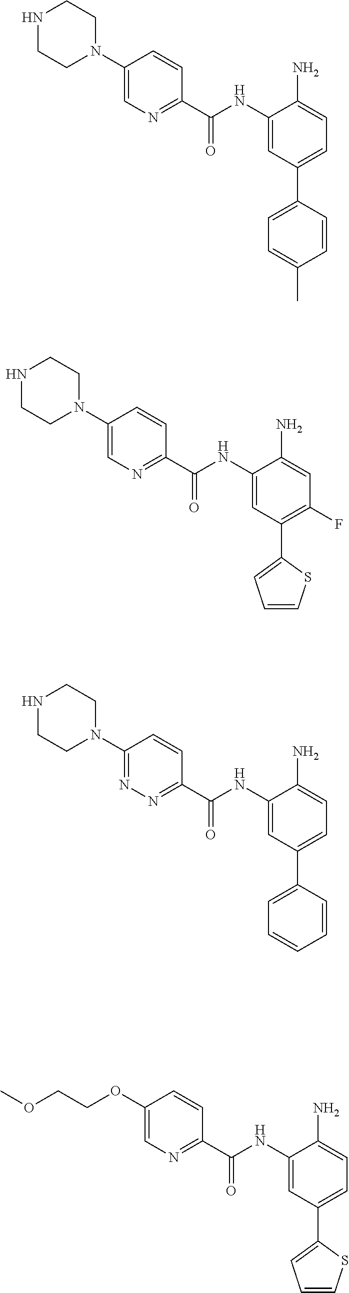 [SMILES: Cc1ccc(-c2ccc(N)c(NC(=O)c3ccc(N4CCNCC4)cn3)c2)cc1 ; Nc1cc(F)c(-c2cccs2)cc1NC(=O)c1ccc(N2CCNCC2)cn1 ; Nc1ccc(-c2ccccc2)cc1NC(=O)c1ccc(N2CCNCC2)nn1 ; COCCOc1ccc(C(=O)Nc2cc(-c3cccs3)ccc2N)nc1] 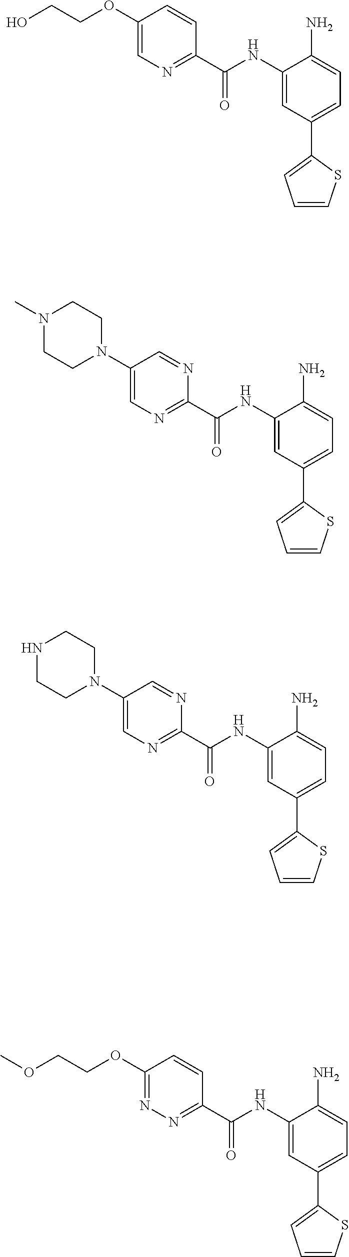 [SMILES: Nc1ccc(-c2cccs2)cc1NC(=O)c1ccc(OCCO)cn1 ; CN1CCN(c2cnc(C(=O)Nc3cc(-c4cccs4)ccc3N)nc2)CC1 ; Nc1ccc(-c2cccs2)cc1NC(=O)c1ncc(N2CCNCC2)cn1 ; COCCOc1ccc(C(=O)Nc2cc(-c3cccs3)ccc2N)nn1] 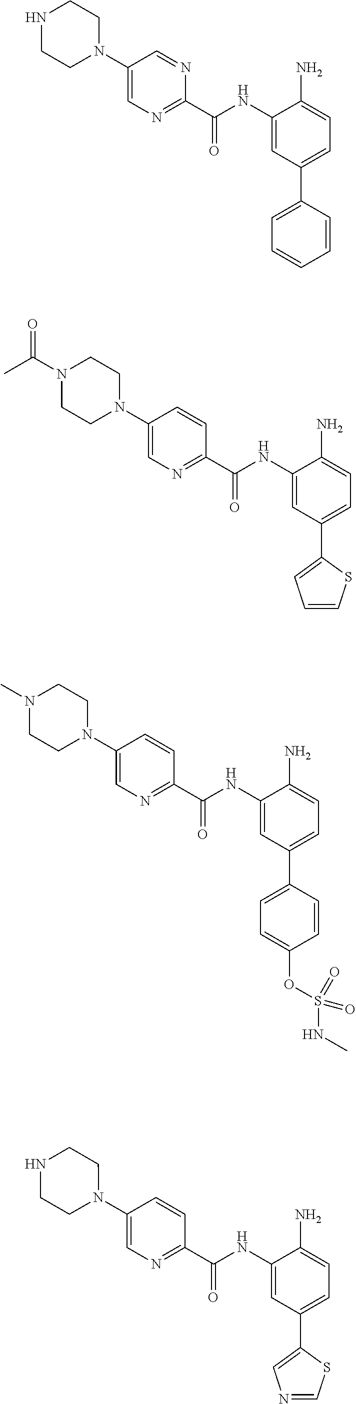 [SMILES: Nc1ccc(-c2ccccc2)cc1NC(=O)c1ncc(N2CCNCC2)cn1 ; CC(=O)N1CCN(c2ccc(C(=O)Nc3cc(-c4cccs4)ccc3N)nc2)CC1 ; CNS(=O)(=O)Oc1ccc(-c2ccc(N)c(NC(=O)c3ccc(N4CCN(C)CC4)cn3)c2)cc1 ; Nc1ccc(-c2cncs2)cc1NC(=O)c1ccc(N2CCNCC2)cn1] 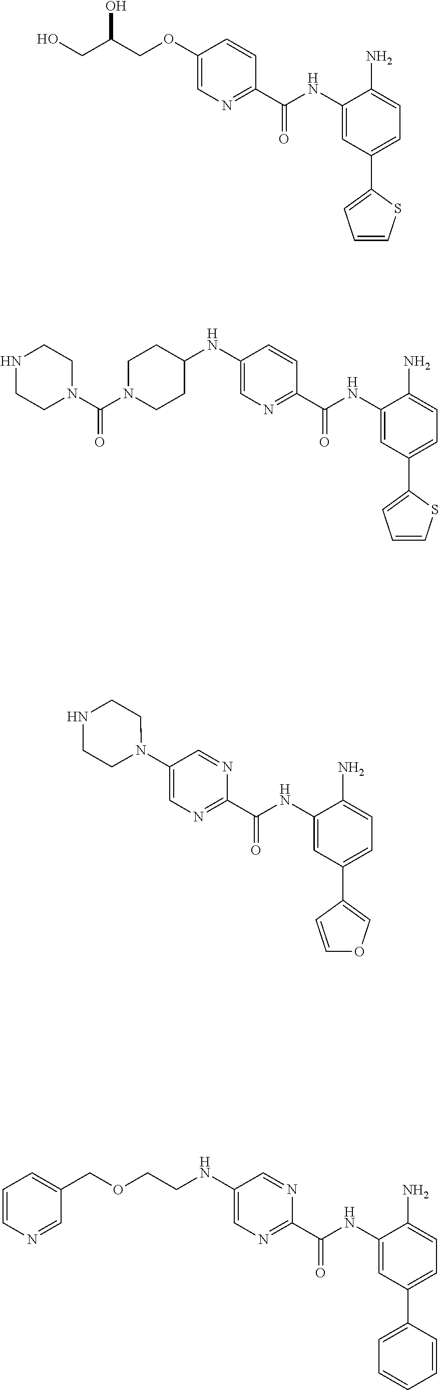 [SMILES: Nc1ccc(-c2cccs2)cc1NC(=O)c1ccc(OC[C@H](O)CO)cn1 ; Nc1ccc(-c2cccs2)cc1NC(=O)c1ccc(NC2CCN(C(=O)N3CCNCC3)CC2)cn1 ; Nc1ccc(-c2ccoc2)cc1NC(=O)c1ncc(N2CCNCC2)cn1 ; Nc1ccc(-c2ccccc2)cc1NC(=O)c1ncc(NCCOCc2cccnc2)cn1] 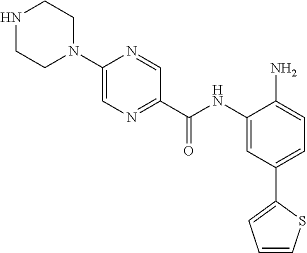 [SMILES: Nc1ccc(-c2cccs2)cc1NC(=O)c1cnc(N2CCNCC2)cn1]
or a pharmaceutical acceptable salt thereof.

11. A pharmaceutical composition comprising a compound of any of claims 1-10, or a pharmaceutically acceptable salt thereof, together with a pharmaceutically acceptable carrier.

12. A method of inhibiting the activity of HDAC1 and/or HDAC2 in a subject of need thereof, comprising administering to the subject a compound of any of claims 1-10, or the pharmaceutical composition of claim 11.

13. A method of treating a disease mediated by HDAC1 and/or HDAC2 in a subject of need thereof, comprising administering to the subject a compound of any of claims 1-10, or the pharmaceutical composition of claim 11.

14. The method of claim 13, wherein the disease is a neurodegenerative disease.

15. The method of claim 14, wherein the neurodegenerative disease is selected from the group consisting of Alzheimer's disease, senile dementia, vascular dementia, Parkinson's disease, Huntington's disease, frontotemporal dementia, and progressive supranuclear palsy.

14. A method of treating a psychological disorder in a subject of need thereof, comprising administering to the subject a compound of any of claims 1-10, or the pharmaceutical composition of claim 11

15. The method of claim 14, wherein the psychological disorder is schizophrenia, major depression, substance use disorder, alcoholism, opiate addiction, cocaine addiction, and post-traumatic stress disorder.

16. A method of treating a cancer in a subject of need thereof, comprising administering to the subject a compound of any of claims 1-10, or the pharmaceutical composition of claim 11.

17. The method of claim 16, wherein the cancer is selected from the group consisting of lung cancer, colon and rectal cancer, breast cancer, prostate cancer, liver cancer, pancreatic cancer, brain cancer, kidney cancer, ovarian cancer, stomach cancer, skin cancer, and bone cancer.

18. The method of claim 16, wherein the cancer is leukemia or lymphoma.

19. The method of claim 16, wherein the cancer is glioblastoma.

20. The method of any of claims 12-19, wherein the subject is human.
